(19) United States
(12) Patent Application Publication
(10) Pub. No.: US 2022/0183938 A1
(43) Pub. Date: Jun. 16, 2022

(54) NANOPARTICLES COMPRISING DEAD SEA EXTRACT AND USES THEREOF

(71) Applicant: AHAVA - DEAD SEA LABORATORIES LTD., Lod (IL)

(72) Inventors: Dror COHEN, D.N the Dead Sea (IL); Ma'or ZE'EVI, D.N Arvot Hayarden (IL); Meital PORTUGAL COHEN, Jerusalem (IL)

(73) Assignee: AHAVA - DEAD SEA LABORATORIES LTD., Lod (IL)

(21) Appl. No.: 17/594,092

(22) PCT Filed: Apr. 5, 2020
(86) PCT No.: PCT/IL2020/050410

§ 371 Date: Oct. 1, 2021

Related U.S. Application Data
(60) Provisional application No. 62/830,468, filed on Apr. 7, 2019.

(51) Int. Cl. A61K 8/11; A61Q 19/00; A61K 8/72; A61K 8/64

(52) U.S. Cl. CPC A61K 8/11; A61Q 19/00; A61K 2800/413; A61K 8/64; A61K 8/72

(57) ABSTRACT

Provided are nanoparticles including at least one Dead Sea Extract, as an active ingredient, and at least one amphipathic material. Further provided are skin-care compositions and/or pharmaceutical compositions including the nanoparticles. Cosmetic and/or therapeutic methods utilizing the nanoparticles are disclosed as well.

### SEQUENCE LISTING

The Sequence Listing submitted in text format (.txt) filed on Sep. 30, 2021, named “SequenceListing.txt”, created on Sep. 13, 2021 (745 bytes), is incorporated herein by reference.

### TECHNOLOGICAL FIELD

This invention relates to nanoparticles comprising Dead Sea extract useful for skin care and skin protection.

### BACKGROUND OF THE INVENTION

The skin is the largest organ of the human body, presenting a total area of close to 2 m². It acts as a barrier between the organism and the external environment. Important skin functions include protection against UV radiation, physical and chemical damage and microbiological attack, maintenance of the body temperature and sensorial functions such as pain and temperature. The skin is mainly composed of two layers, the epidermis and the dermis, besides the subcutaneous tissue. It is composed of a variety of different cells. The epidermis is composed of several lipids including phospholipids, phosphatidylcholine, cholesterol and triglycerides. The main cell types found in the epidermis are keratinocytes, melanocytes, Langerhans cells, and Merkel cells. The epidermis is divided into several layers and its outermost layer, the stratum corneum, is responsible for the barrier function of the skin due to its lipophilicity and high cohesion between cells. The stratum corneum is composed of keratinized corneocytes embedded in lipid bilayers. Ceramides, cholesterol, and free fatty acids comprise its extracellular lipid compartment. The dermis is the layer next to the subcutaneous tissue and it is composed of collagen, elastin, glycosaminoglycans and fibroblasts. This layer is highly vascularized besides containing the appendices (sweat glands and pilosebaceous units) and leucocytes, adipocytes and mast cells. Physical changes occur through the skin layer. The two main examples are temperature and pH. The temperature of the skin is at around 32° C. but increases after passing the layers to reach body temperature in the dermis layer at around 37° C. Similarly, the pH of the skin varies from its surface into deeper layers. pH of healthy skin is slightly acidic to fight against microbial contamination and is comprised between 4.5<pH<5.5. However, the intercellular pH after passing the stratum corneum is around 6.2-6.5. Then, pH is reaching its physiological value (pH 7.4) in the deeper layer of the epidermis before reaching the dermis layer.

The stratified arrangement of the epidermis is maintained by the continual production and differentiation of keratinocytes, a process that must be tightly regulated to ensure that the epidermal barrier remains functional as it continues to evolve. Accordingly, the stepwise maturation of keratinocytes is a carefully choreographed routine that is guided by converging signals from a myriad of interconnected pathways. Proteases and their inhibitors are rapidly emerging as significant contributors throughout this critical operation. Skin conditions usually results in an overexpression of one or more proteases, resulting in the rupture of the fragile equilibrium in the stratum corneum. This loss of equilibrium affects skin barriers and usually generates an inflammation of the skin. This inflammation is responsible to an increase of pH of the skin debilitating its protector. Considering the skin anatomy and physiology, some active substances will not provide the desired activity after their cutaneous administration.

Nanotechnology is used to modify the drug permeation/penetration by controlling the release of active substances and increasing the period of permanence on the skin besides ensuring a direct contact with the stratum corneum and skin appendices and protecting the drug against chemical or physical instability. The potential cosmetic use of nano-particles such as nano-capsules was investigated in the early 1990s [1]. Since then, preparation methods have been developed along with studies regarding the supramolecular structures of such nanoparticles [2]-[7].

Dead Sea water, salts, minerals and mud are well known for their therapeutic efficacy in treating a variety of skin conditions such as psoriasis, atopic dermatitis, acne and other inflammation skin diseases as well as for their cosmetic benefits [8]-[11].

### REFERENCES

[1] WO 9305753.
[2] Hanson, J. A.; Deming, T. J. Polym. Chem. 2011, 2, 1473-1475.
[3] Hanson, J. A.; Chang, C. B.; Graves, S. M.; Li, Z.; Mason, T. G.; Deming, T. J. Nature 2008, 455, 85-88.
[4] C. Charcosset, Chapter 6: Membranes for the Preparation of Emulsions and Particles, in Membrane Processes in Biotechnology and Pharmaceutics, (Ed. C. Charcosset), Elsevier, 2012, pp. 213-251.
[5] Braunecker, W. A.; Matyjaszewski, K. Prog. Polym. Sci. 2007, 32, 93-146.
[6] Bacinello, D.; Garanger, E.; Taton, D.; Tam, K. C.; Lecommandoux, S. Biomacromelcules 2014, 15, 1882-1888.
[7] Lecommandoux, S.; J. Am. Chem. Soc. 2005, 127, 7, 2026-2027.
[8] Sukenik S., et al., Treatment of psoriatic arthritis at the Dead Sea. J. Rheumatol. 1994, 21, 1305-1309.
[9] S. Halevy., et al. Dead Sea bath salt for the treatment of psoriasis vulgaris: a double-blind controlled study. Journal of the European Academy of Dermatology and Venereology, 1997, 9, 237-242.
[10] Maor Z. and Yehuda S. Skin smoothing effects of Dead Sea minerals: comparative profilometric evaluation of skin surface. International Journal of Cosmetic Science, 1997, 19, 105-110.
[11] Shimon W. Moses, Michael David, Ehud Goldhammer, Asher Tal and Shaul Sukenik. The Dead Sea, A Unique Natural Health Resort. IMAJ, 2006, 8, 483-488.

Acknowledgement of the above references herein is not to be inferred as meaning that these are in any way relevant to the patentability of the presently disclosed subject matter.

### SUMMARY OF THE INVENTION

The present invention discloses a delivery system configured to deliver active ingredients such as Dead Sea salts and/or minerals to at least a region of the skin. The delivery system is based on stimuli-responsive nanoparticles that are capable of releasing the active ingredients upon changes in pH and/or in the presence of enzymes encountered in the skin conditions targeted (e.g., inflamed skin cells).

The stimuli-response nanoparticles, which may also be referred to as “smart” and/or “environmentally sensitive” nanoparticles, are comprised of a polymeric amphipathic material that is capable of responding to change/s in physiological stimuli such as pH and/or presence of enzymes and other biomolecules by undergoing dissociation which results in the release of the active ingredient e.g., at the site of interest.

Thus, in one of its aspects the present invention provides a nanoparticle comprising at least one Dead Sea Extract, as an active ingredient, and at least one amphipathic material of the form A-L-B, wherein A is an hydrophilic polymer, B is an hydrophobic polymer and L is a linker segment or a chemical bond associating A to B.

In the at least one amphipathic material disclosed herein above and below, A and B may be oppositely oriented around L i.e., the amphipathic material may be represented by the form B-L-A. Such an amphipathic material is within the scope of the invention disclosed herein above and below.

Hence, in some embodiments the at least one amphipathic material disclosed herein above and below is an amphipathic material of the form A-L-B or B-L-A, wherein A is an hydrophilic polymer, B is an hydrophobic polymer and L is a linker segment or a chemical bond associating between A and B.

In another one of its aspects the present invention provides a nanoparticle comprising at least one Dead Sea Extract, as an active ingredient, and at least one amphipathic material of the form A-L-B or B-L-A, wherein A is an hydrophilic, B is an hydrophobic polymer and L is a linker segment or a chemical bond associating between A and B, wherein a combination of A, L and B in the material of the form A-L-B or B-L-A is configured as an amphipathic material for forming one or more of a nanocarrier, a nanocapsule, a delivery system and an arraignment as disclosed herein.

In a further one of its aspects the present invention provides a nanoparticle comprising at least one Dead Sea Extract, as an active ingredient, and at least one amphipathic material of the form A-L-B and/or B-L-A, wherein A is an hydrophilic polymer, B is an hydrophobic polymer and L is a linker segment or a chemical bond associating between A and B, wherein a combination of A, L and B in the material of the form A-L-B or B-L-A is configured as an amphipathic material for forming one or more of a nanocarrier, a nanocapsule, a delivery system and an arraignment as disclosed herein, and wherein the amphipathic material is configured to undergo dissociation when in contact with skin environment having one or more of: (i) pH of between about 6.2 to 7.4; (ii) presence of one or more of matrix metalloproteinase MMP2 and MMP9; and (iii) presence of at least one Cathepsin B enzyme, to thereby release the active ingredient to the skin environment.

In yet another one of its aspects the present invention provides an nanocarrier comprising at least one amphipathic material of the form A-L-B or B-L-A, wherein A is an hydrophilic polymer, B is an hydrophobic polymer and L is a linker segment or a chemical bond associating between A and B, wherein said nanocarrier comprises at least one Dead Sea Extract as an active ingredient.

In a further one of its aspects the present invention provides a nanocapsule comprising a core and a shell material, wherein the core comprises at least one Dead Sea Extract, as an active ingredient, and the shell material comprises at least one amphipathic material of the form A-L-B or B-L-A, wherein A is an hydrophilic polymer, B is an hydrophobic polymer and L is a linker segment or a chemical bond associating between A and B.

In yet a further one of its aspects the present invention provides an emulsion comprising a plurality of the nanoparticles according to the invention.

Yet in a further one of its aspects the present invention provides an arraignment comprising a plurality of amphipathic materials around a core, wherein the core comprises at least one Dead Sea Extract, as an active ingredient, and wherein the plurality of amphipathic materials form a shell material substantially surrounding said core, said amphipathic materials each being of the form A-L-B or B-L-A, wherein A is an hydrophilic polymer, B is an hydrophobic polymer and L is a linker segment or a chemical bond associating between A and B.

In another one of its aspects the present invention provides delivery system for the delivery of at least one Dead Sea Extract to at least one skin region (e.g., inflammable skin region) wherein the delivery system comprises the nanoparticle according to the invention.

In a further one of its aspects the present invention provides delivery system for the delivery of at least one Dead Sea Extract to at least one skin region (e.g., inflammable skin region) wherein the delivery system comprises the emulsion according to the invention.

In yet a further one of its aspects the present invention provides delivery system for the delivery of at least one Dead Sea Extract to at least one skin region (e.g., inflammable skin region) wherein the system comprises a nanoparticle comprising at least one Dead Sea Extract, as an active ingredient, and at least one amphipathic material of the form A-L-B or B-L-A, wherein A is an hydrophilic polymer, B is an hydrophobic polymer and L is a linker segment or a chemical bond associating between A and B, wherein the amphipathic material is selected to undergo dissociation and/or wherein the linker L is selected to undergo dissociation from either polymer A and/or polymer B when in contact with skin environment having one or more of: (i) pH of between about 6.2 to 7.4; (ii) presence of one or more of matrix metalloproteinase MMP2 and MMP9; and (iii) presence of at least one Cathepsin B enzyme, wherein said dissociation induced the degradation of the amphipathic material and the release of the active ingredient to said skin environment.

In a further one of its aspects the present invention provides delivery system for the delivery of at least one Dead Sea Extract to at least one skin region (e.g., inflammable skin region) wherein the system comprises a nanocapsule comprising a core and a shell material, wherein the core comprises at least one Dead Sea Extract, as an active ingredient, and the shell material comprises at least one amphipathic material of the form A-L-B or B-L-A, wherein A is an hydrophilic polymer, B is an hydrophobic polymer and L is a linker segment or a chemical bond associating between A and B, wherein the amphipathic material is selected to undergo dissociation and/or wherein the linker L is selected to undergo dissociation from either polymer A and/or polymer B when in contact with skin environment having one or more of: (i) pH of between about 6.2 to 7.4; (ii) presence of one or more of matrix metalloproteinase MMP2 and MMP9; and (iii) presence of at least one Cathepsin B enzyme, wherein said dissociation induced the degradation of the shell material and the release of the active ingredient from the core to said skin environment.

In a further one of its aspects the present invention provides a composition comprising the nanoparticle according to the invention.

In yet a further one of its aspects the present invention provides a composition comprising the delivery system according to the invention.

In another one of its aspects the present invention provides skin-care compositions (formulations) and/or pharmaceutical compositions (formulations) comprising the nanoparticles and/or delivery system and/or arraignment according to the invention.

In yet another one of its aspects the present invention provides the nanoparticles and/or delivery system and/or arraignment according to the invention for use in the preparation of skin-care and/or pharmaceutical formulations.

In a further one of its aspects the present invention provides the nanoparticles and/or compositions and/or formulations and/or delivery system and/or emulsion according to the invention for one or more of protecting and/or improving the state of the skin, and preventing and/or treating imperfections of the skin of a subject.

Yet, in a further one of its aspects the present invention provides the nanoparticles and/or compositions and/or formulations and/or delivery system and/or arraignment and/or emulsions according to the invention for treating or preventing at least one disease or disorder of the skin.

In a further one of its aspect the present invention provides the use of the nanoparticle and/or delivery system and/or arraignment according to the invention for the preparation of a pharmaceutical composition for treating or preventing a disease or disorder of the skin.

In another one of its aspects the present invention provides one or more of a lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, and an eye make-up comprising the nanoparticles and/or compositions and/or formulations and/or delivery system/ and/or emulsions according to the invention.

Yet, in a further one of its aspects the present invention provides a method for one or more of protecting and/or improving the state of the skin of a subject and preventing and/or treating imperfections of the skin of a subject in need thereof, wherein the method comprises topically administering the nanoparticles and/or compositions and/or formulations and/or delivery system and/or arraignment and/or emulsions according to the invention onto the skin of the subject.

In another one of its aspects the present invention provides a method for treating or preventing a disease or disorder of the skin of a subject (at times treating or preventing or reducing irritation and/or inflammation associate with the disease or disorder), the method comprises topically administering to a subject in need thereof a nanoparticles and/or compositions and/or formulations and/or delivery system and/or arraignment according to the invention.

In a further one of its aspects the present invention provides a method for treating and/or preventing one or more disease or disorder, the method comprises topical administration of the nanoparticles and/or compositions and/or formulations and/or delivery system and/or arraignment and/or emulsions according to the invention to a subject in need thereof, wherein the disease or disorder are associated with skin environment having one or more of (i) pH of between about 6.2 to 7.4; (ii) presence of one or more of matrix metalloproteinase MMP2 and MMP9; and (iii) presence of at least one Cathepsin B enzyme.

In a further one of its aspects the present invention provides a method for treating and/or preventing one or more disease or disorder, the method comprises topical administration of the nanoparticles and/or compositions and/or formulations and/or delivery system and/or arraignment and/or emulsions according to the invention to a subject in need thereof, wherein the disease or disorder are selected from irritant contact dermatitis (ICD), allergic contact dermatitis (ACD), skin photo-aging and skin pigmentation.

Yet, in a further one of its aspects the present invention provides a method for the encapsulation of at least one Dead Sea Extract, the method comprising:

providing a water solution comprising at least one Dead Sea Extract and optionally at least one amphipathic material substantially as disclosed herein;
providing an oil solution comprising at least one oil and optionally at least one amphipathic material substantially as disclosed herein;
provided that one of said water solution or oil solution comprises the at least one amphipathic material and being at a pH of between about 4.0 to about 6.0; and

applying means to disperse the aqueous solution into the oil solution to thereby obtain a nanocapsule comprising a core and a shell, wherein said core comprises the at least one Dead Sea Extract and wherein said shell comprises the at least one amphipathic material which is substantially continuously assembled around the core.

Yet, in a further one of its aspects the present invention provides a method for the encapsulation of at least one Dead Sea Extract, the method comprising:

providing a water solution comprising at least one Dead Sea Extract and an amphipathic material of the form A-L-B or B-L-A, wherein A is an hydrophilic polymer, B is an hydrophobic polymer and L is a linker segment or a chemical bond associating between A and B, wherein said aqueous solution is at a pH of between about 4.0 to about 6.0;

providing an oil solution comprising at least one oil; and

applying means to disperse the aqueous solution into the oil solution to thereby obtain a nanocapsule comprising a core and a shell, wherein said core comprises the at least one Dead Sea Extract and wherein said shell comprises the amphipathic material which is substantially continuously assembled around the core.

Yet, in a further one of its aspects the present invention provides a method for the encapsulation of at least one Dead Sea Extract, the method comprising:

providing a water solution comprising at least one Dead Sea Extract;

providing an oil solution comprising an amphipathic material of the form A-L-B or B-L-A, wherein A is an hydrophilic polymer, B is an hydrophobic polymer and L is a linker segment or a chemical bond associating between A and B, wherein said oil solution is at a pH of between about 4.0 to about 6.0; and

applying means to disperse the aqueous solution into the oil solution to thereby obtain a nanocapsule comprising a core and a shell, wherein said core comprises the at least one Dead Sea Extract and wherein said shell comprises the amphipathic material which is substantially continuously assembled around the core.

The present invention also provides nanoparticles, nanocarriers, nanocapsules, delivery systems, arraignments, emulsions, compositions, extracts, uses and methods as herein described and/or exemplified.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 provides a schematic representation of the nanocapsules according to some embodiments of the invention.

FIG. 2 provides a schematic representation of the assembly of the amphipathic material with adjusted Hydrophilic/Lipophilic Balance (HLB) to stabilize water in oil (W/O) or oil in water (O/W) emulsions, according to some embodiments of the invention.

FIG. 3A-3B provides a schematic representation of the synthesis of poly(ethylene glycol)-block-poly(γ-benzyl-L-glutamate-co-L-valine) (FIG. 3A) and the synthesis of the amphipathic material poly(ethylene glycol)-block-poly(L-glutamate-co-L-valine) (FIG. 3B), according to some embodiments of the invention.

FIG. 4 provides a schematic representation of the synthesis of an amphipathic material according to some embodiments of the invention.

FIG. 5 provides a schematic illustration of the effect of topical application of Dead Sea extract (Osmoter) encapsulated in nanocapsules (NC), of naked NC (with no active ingredient) and of Dead Sea extract (Osmoter) alone, on the viability of human skin after UVB radiation, according to some embodiments of the invention.

FIG. 6 provides a schematic illustration of the effect of topical application of Dead Sea extract (Osmoter) encapsulated in NC, of naked NC (with no active ingredient) and of Dead Sea extract (Osmoter) alone, on apoptosis activity (Caspase 3 activity) of human skin after UVB radiation, according to some embodiments of the invention.

FIG. 7 provides a schematic illustration of the effect of topical application of Dead Sea extract (Osmoter) encapsulated in NC, of naked NC (with no active ingredient) and of Dead Sea extract (Osmoter) alone, on secretion of TNFα by human skin after UVB radiation, according to some embodiments of the invention.

### DETAILED DESCRIPTION OF THE INVENTION

In a first one of its aspects the present invention provides a nanoparticle comprising at least one Dead Sea Extract and at least one amphipathic material of the form A-L-B or B-L-A, wherein:

A is an hydrophilic polymer;

B is an hydrophobic polymer; and

L is a linker segment or a chemical bond associating between A and B.

Various embodiments will be detailed herein in connection with the aforementioned first aspect of the invention. It is noted that one or more of these embodiments may be applicable to one or more aspects of the invention disclosed herein above and below e.g., nanocarriers, nanocapsules, arraignments, delivery systems, compositions, uses, emulsions and methods.

It is further noted that one or more embodiments which are detailed in connection with one aspect of the invention may also be applicable to other one or more aspects of the invention.

The term “nanoparticle” according to the present invention refers to a material in a particulate form that may be in the form of a nanocarrier or a nanocapsule.

In general, the nanoparticles of the invention are polymeric based nanoparticles. In particular, they are comprised of polymeric amphipathic material/s.

In some embodiments the nanoparticle is a nanocarrier comprising at least one amphipathic material of the form A-L-B or B-L-A, wherein A is an hydrophilic polymer, B is an hydrophobic polymer and L is a linker segment or a chemical bond associating between A and B, wherein the nanocarrier comprises at least one Dead Sea Extract as an active ingredient.

As used herein, the term “nanocarrier” refers to a material in a particulate form that comprises the Dead Sea extract (active ingredient) either in the material polymeric matrix making up the nanoparticle (e.g., the active material may be absorbed in and/or adsorbed to and/or mixed with the material matrix) or in its core (e.g., a core/shell or pseudo core/shell type nanoparticle).

In some embodiments the nanocarrier may further comprise at least one another material such as a carrier, diluent, an excipient, a surfactant and the like, or any combination thereof.

The at least one another material may be a non-active and/or an active material.

In some embodiments the at least one another material may be a steric stabilizing agent.

In some embodiments the at least one another material is substantially soluble in water.

In some embodiments the at least one another material is hydrophilic.

In some embodiments the nanoparticle is a nanocapsule comprising a core and a shell material, wherein the core comprises at least one Dead Sea Extract, as an active ingredient, and the shell material comprises at least one amphipathic material of the form A-L-B or B-L-A, wherein A is an hydrophilic polymer, B is an hydrophobic polymer and L is a linker segment or a chemical bond associating between A and B.

As used herein, the term “nanocapsule” refers to a nanoparticle form having a shell of a material and a core, the core comprising the Dead Sea extract (active ingredient). The shell is comprised of a continuous material that is assembled around the core and surrounding the outmost surface of the active ingredient and the latter being encapsulated therein. In particular, the shell is comprised of the polymeric amphipathic material disclosed herein.

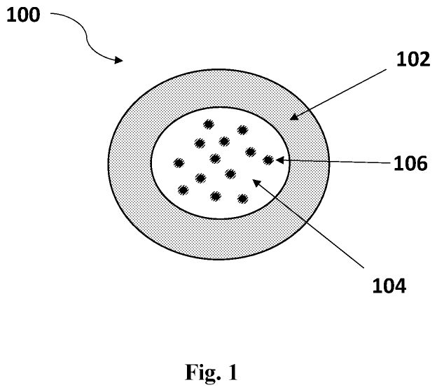

FIG. 1 provides a non-limiting schematic representation of the nanocapsules (100) according to some embodiments of the invention. The nanocapsules have a substantially spherical structure, with a polymeric shell (102) comprising the amphipathic material according to the invention and with a core (104) comprising a Dead Sea extract (106) e.g., Dead Sea salts/minerals. It is noted that the dimensions of each component of the nanocapsule in FIG. 1 and the relation between same are not to be considered as limiting.

In some embodiments the core of the nanocapsule may optionally further comprise at least one another material such as a carrier, diluent, an excipient, a surfactant and the like, or any combination thereof.

In some embodiments the at least one another material is substantially soluble in water.

In some embodiments the at least one another material is hydrophilic.

In some embodiments the at least one another material may be a non-active and/or an active material.

In some embodiments the core of the nanoparticle is an aqueous core and the Dead Sea extract is diluted therein.

In some embodiments the Dead Sea extract is present in the core of the nanoparticle in its pure form e.g., as is.

In some embodiments the Dead Sea extract forms the core of the nanoparticle.

In some embodiments the core of the nanoparticle (e.g., nanocapsule) is in a form of an hydrophilic droplet.

In some embodiments the core of the nanoparticle (e.g., nanocapsule) constitutes a single drop comprising the Dead Sea extract (e.g., Dead Sea water).

In some embodiments the core of the nanoparticle (e.g., nanocapsule) constitutes a single drop of the Dead Sea extract (e.g., Dead Sea water).

In some embodiments the shell of the nanocapsule may optionally further comprise at least one another material such as a carrier, diluent, an excipient, a surfactant.

In some embodiments the shell of the nanocapsule may optionally further comprise at least one steric stabilizing agent.

The nanoparticle according to the invention may be of any shape and size, provided that the size dimensions thereof e.g., diameter, length, width, thickness are at the nanoscale.

In some embodiments according to the invention the nanoparticle has a colloidal structure.

In some embodiments according to the invention the nanoparticle has a substantially spherical shape.

Generally, the nanoparticle according to the invention is of a size (diameter or longest axis) of between about 1 to about 200 nm, or any size there between. In some embodiments, the size is between 1 to 200 nm, between 1 to 150 nm, between 1 to 100 nm, between 10 to 200 nm, between 10 to 150 nm, between 10 to 100 nm, between 20 to 200 nm, between 20 to 150 nm, between 20 to 100 nm, between 40 to 200 nm, between 40 to 150 nm, or between 40 to 100 nm. Any value which is between any one of the above values is within the scope of the present disclosure.

In some embodiments, the size (diameter or longest axis) of the nanoparticle according to the invention is about 200 nm, about 190 nm, about 180 nm, about 170 nm, about 160 nm, about 150 nm, about 140 nm, about 130 nm, about 120 nm, about 100 nm, or about 90 nm.

In some embodiments, the size (diameter or longest axis) of the nanoparticle according to the invention is between about 10 to 150 nm, between about 20 to 150 nm, between about 30 to 150 nm, between about 40 to 150 nm, between about 50 to 150 nm, between about 60 to 150 nm, between about 70 to 150 nm, between about 80 to 150 nm, between about 90 to 150 nm, between about 100 to 150 nm, between about 10 to 100 nm, between about 20 to 100 nm, between about 30 to 100 nm, between about 40 to 100 nm or between about 50 to 100.

In some embodiments, the size of the nanoparticle according to the invention is between about 40 to 150 nm or between about 40 to 100 nm with narrow size distribution.

In some embodiments, the nanoparticle is a spherical nanoparticle and the diameter thereof is of the size described herein.

In some embodiments, the diameter of the nanoparticle is an hydrodynamic diameter.

As may be appreciated by a person versed in the art, the size of the nanoparticles according to the present invention may be determined by any known techniques. Non limiting example of such technique is Dynamic Light Scattering (DLS) e.g., utilizing Zetasizer Nano ZS at 25° C.

In some embodiments, the nanoparticles according to the invention may act via the skin with not penetration into the blood stream.

In some embodiments according to the invention the nanoparticles of the present invention have a narrow size distribution e.g., ±10%, at times ±5%, even at times ±1%.

As used herein the terms “amphipathic material” or “amphiphilic material” refer to a polymeric material possessing both hydrophilic (water-loving, polar) and lipophilic (fat-loving) properties. The hydrophilic and/or lipophilic nature of the material may be determined based on one or more of the nature of the units building the polymeric material, the number of the units (e.g., repeating units), the presence of functional groups in the polymeric material, the number of functional groups and the like. The hydrophilic and/or lipophilic nature of the material may also be affected from the surrounding pH.

In some embodiments in the amphipathic material of the form A-L-B or B-L-A a combination of A, L and B is configured as an amphipathic material for forming stable one or more of the nanocarrier, nanocapsule, delivery system and the arraignment according to the invention.

In some embodiments the nanoparticles are stable at a pH of between about 4.0 (inclusive) to about 6.0 (inclusive). Any value which is between any one of pH 4.0 and pH 6.0 is within the scope of the present disclosure.

In some embodiments the nanoparticle according to the invention are stable at pH of 4.0, 4.1, 4.2, 4.3, 4.4, 4.5, 4.6, 4.7, 4.8, 4.9, 5.0, 5.1, 5.2, 5.2, 5.3, 5.4, 5.5, 5.6, 5.7, 5.8, 5.9 or 6.0. In some embodiments the nanoparticle according to the invention are stable at pH of about 5.5.

In some embodiments in the amphipathic material of the form A-L-B or B-L-A, a combination of A, L and B in said amphipathic material is configured to provide an amphipathic material, wherein said amphipathic material is configured to undergo dissociation in environment having one or more of: (i) pH of between about 6.2 to 7.4; (ii) presence of one or more of matrix metalloproteinase MMP2 and MMP9; and (iii) presence of at least one Cathepsin B enzyme.

In some embodiments said environment is a skin environment.

In some embodiments, a combination of A, L and B in the material of the form A-L-B or B-L-A is configured as an amphipathic material for forming one or more of a nanocarrier, a nanocapsule, a delivery system and an arraignment as disclosed herein. The amphipathic material is further configured to undergo dissociation when in contact with skin environment having one or more of: (i) pH of between about 6.2 to 7.4; (ii) presence of one or more of matrix metalloproteinase MMP2 and MMP9; and (iii) presence of at least one Cathepsin B enzyme, to thereby release the active ingredient to the skin environment.

In some embodiments the amphipathic material is configured to undergo dissociation when in contact with skin environment having one or more of: (i) pH of between about 6.2 (inclusive) to about 7.4 (inclusive); (ii) presence of one or more of matrix metalloproteinase MMP2 and MMP9; and (iii) presence of at least one Cathepsin B enzyme.

In some embodiments the amphipathic material is configured to undergo dissociation when in contact with skin environment having pH of between about 6.2 to about 7.4. Any value which is between any one of about pH 6.2 and about pH 7.4 is within the scope of the present disclosure.

In some embodiments the amphipathic material is configured to undergo dissociation when in contact with skin environment having pH of 6.2, 6.3, 6.4, 6.5, 6.6, 6.7, 6.8, 6.9, 7.0, 7.1, 7.2, 7.3 or 7.4.

In some embodiments the amphipathic material is configured to undergo dissociation when in contact with skin environment having presence of one or more of matrix metalloproteinase MMP2 and MMP9.

In some embodiments the amphipathic material is configured to undergo dissociation when in contact with skin environment having presence of at least one Cathepsin B enzyme.

The amphipathic material according to the invention forms part of the nanoparticle according to the invention.

In some embodiments the amphipathic material is a self-assembled material forming a continuous structure.

In some embodiments the amphipathic material is assembled to form a continuous structure.

In some embodiments the amphipathic material is present in the nanoparticle in the form of a shell.

In some embodiments the amphipathic material is assembled around a core to provide a continuous shell around the core.

In some embodiments the amphipathic material are assembled via nano-emulsion techniques know in the art (e.g., See examples below).

As noted above, the amphipathic material according to the present invention is of the form A-L-B or B-L-A, wherein:

A is an hydrophilic polymer;

B is an hydrophobic polymer; and

L is a linker segment or a chemical bond associating between A and B.

In some embodiments A is a linear polymer.

In some embodiments A is a branched polymer.

In some embodiments A is a block polymer.

In some embodiments A is a homo-polymer.

In some embodiments A is a copolymer.

In some embodiments A is poly(ethylene glycol) (PEG) [a hydrophilic block at low pH e.g., pH of between about 4.0 (inclusive) to about 6.0 (inclusive). At times at pH of about 5.5].

In some embodiments B is a linear polymer.

In some embodiments B is a branched polymer.

In some embodiments B is a block polymer.

In some embodiments B is a homo-polymer.

In some embodiments B is a copolymer.

In some embodiments B is a diblock polymer.

In some embodiments B is polyglutamic acid (PGA) polymer [a hydrophobic block at low pH e.g., of between about 4.0 (inclusive) to about 6.0 (inclusive). At times at pH of about 5.5].

In some embodiments A is a block polymer and B is a block polymer.

In some embodiments A is a block polymer and B is a di-block polymer.

In some embodiments the amphipathic material is a copolymer.

In some embodiments the amphipathic material is a diblock copolymer e.g., wherein there is a direct bond between A (the hydrophilic polymer) and B (the hydrophobic polymer).

At times, wherein there is a direct bond between A and B, L is referred to herein as absent.

In some embodiments the amphipathic material is a diblock copolymer wherein the blocks are connected by L which is a peptide.

As used herein the term “copolymer” refers to a polymer derived from more than one species of monomer.

As used herein the term “diblock copolymer” is a polymer consisting of two types of monomers. The monomers are arranged such that there is a chain of each monomer, and those two chains are grafted together to form a single copolymer chain.

Non limiting example of a diblock co-polymer amphipathic material is a polymer wherein A is PEG and B is polyglutamic acid PGA, referred to herein as PEG-PGA.

In some embodiments B is a copolymer.

In some embodiments B is a copolymer of glutamic acid (GA) and another hydrophobic amino acid.

In some embodiments B is a di-block copolymer of glutamic acid (GA) and another hydrophobic amino acid. Non limiting example of such B copolymer is a copolymer of glutamic acid (GA) and Valine (VAL) referred to herein as P(GA-VAL).

In some embodiments the copolymer of GA and VAL may be a diblock co-polymer.

In some embodiments A is a block polymer and B is a di-block copolymer.

In some embodiments A is a block polymer of PEG and B is a di-block copolymer of glutamic acid (GA) and another hydrophobic amino acid.

In some embodiments A is a block polymer of PEG and B is a di-block copolymer of glutamic acid (GA) and VAL.

The molecular weight and hence the number of repeating units or degree of polymerization in the polymers of the amphipathic material according to the invention are selected to provide Hydrophilic/Lipophilic Balance (HLB) that is configured to stabilize the structure of the nanoparticles according to the invention and/or the emulsions comprising thereof e.g., W/O emulsions.

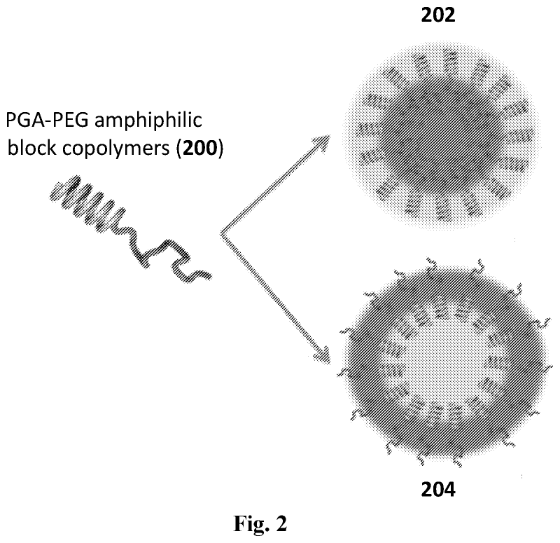

FIG. 2 provides a schematic representation of the assembly of the amphipathic material PGA-PEG amphiphilic block copolymer (200) with Hydrophilic/Lipophilic Balance (HLB) control to stabilize water in oil (W/O) emulsion (202) or oil in water (O/W) emulsion (204) according to some embodiments of the invention.

Without wishing to be bound by theory, the hydrophilic polymer A and the hydrophobic polymer B in the amphipathic material of the invention exhibit complementary action. Coalescence is avoided by the steric stabilization of the “external” block (this “external” block is the one in direct contact with the continuous medium, the hydrophilic one in the case of O/W emulsions and the hydrophobic one in the case of W/O emulsions). Oswald ripening is avoided by the combined effect of the reduction of the interfacial tension between the dispersed and the continuous phases, in addition with the reduction of the chemical potential of the dispersed phase by adsorption of the “internal” block at the O/W interface. The complementary role of both blocks makes the amphipathic material of the invention to produce highly stable nanoparticles and the active ingredients to be efficiently protected from possible degradation mechanism (e.g., aerial oxidation or photo-degradation).

In some embodiments the number of the repeating units of the A polymer is at least 4.

In some embodiments the number of the repeating units of the A polymer is between about (inclusive) 4 to about 100 (inclusive). At times it is between about 4 (inclusive) to about 120 (inclusive). Even at times it is between about 10 (inclusive) to about 50 (inclusive). Any value which is between any one of the above values is within the scope of the present disclosure.

In some embodiments the number of the repeating units of the B polymer is at least 5.

In some embodiments the number of the repeating units of the B polymer is between about 20 (inclusive) to about 100 (inclusive). At times it is between about 20 (inclusive) to about 160 (inclusive). Event at times it is between about 40 (inclusive) to about 100 (inclusive). Any value which is between any one of the above values is within the scope of the present disclosure.

In some embodiments A is PEG and the number of the repeating units of the PEG polymer is between about 4 (inclusive) to about 100 (inclusive). At times it is between about 4 (inclusive) to about 120 (inclusive). Even at times it is between about 10 (inclusive) to about 50 (inclusive). Any value which is between any one of the above values is within the scope of the present disclosure.

In some embodiments the number the repeating units of the PEG polymer is 44.

In some embodiments the number the repeating units of the PEG polymer is about 114.

In some embodiments B is PGA and the number of the repeating units of the PGA polymer is between about 20 (inclusive) to about 100 (inclusive). At times between about 20 (inclusive) to about 160 (inclusive). Even at times between about 40(inclusive) to about 100 (inclusive). Any value which is between any one of the above values is within the scope of the present disclosure.

In some embodiments the number repeating units of the PGA polymer is 46.

In some embodiments the number repeating units of the PGA polymer is 150.

In some embodiments the number repeating units of the PGA polymer is 151.

In some embodiments the number of VAL repeating units in the copolymer P(GA-VAL) is at least about 30% of the number of the GA units of said copolymer.

In some embodiments B is a diblock co-polymer of GA and VAL and the number of VAL repeating units in the copolymer is at least about 30% of the number of the GA units of the diblock co-polymer.

According to the present invention, L is a linker segment or a chemical bond associating between A and B in the amphipathic material according to the present invention.

In some embodiments L may be a non-active linker molecule.

In some embodiments L may be a chemical bond directly connecting between A and B. Non limiting examples of such bonds are C—C single bond, C—N single bond, amide bond and the like. The atoms taking part in said bonds are originated from A and/or B.

In some embodiments L is a peptide unit comprising one or more amino acids, each of which may be optionally substituted.

In some embodiments L is a peptide unit connected to A and B via an amide bond formed between the N or C terminus of the peptide with either A or B.

In some embodiments L is a peptide unit connected to A and/or B via a side chain in the peptide.

In some embodiments L is a peptide unit connected to A and/or B via a substituted side chain in the peptide.

In some embodiments L is the peptide PVGLIG (also referred to herein as SEQ ID No. 1).

In some embodiments L is the peptide PVGLIG, wherein the peptide is either connected to A via the peptide N terminus and to B via the peptide C terminus or wherein the peptide is connected to B via the peptide N terminus and to A via the peptide C terminus.

In some embodiments L is the peptide PVGLIG wherein the peptide is connected to A via the peptide N terminus and to B via the peptide C terminus.

In some embodiments L is the peptide PVGLIG wherein the peptide is connected to B via the peptide N terminus and to A via the peptide C terminus.

In some embodiments L is the peptide βA-PVGLIG-βA-C (also referred to herein as SEQ ID No. 2), wherein C (Cysteine) is optionally substituted and wherein βA is β-Alanine. To this end, L may be connected to B via a peptide bond and to A via the substituted Cysteine moiety.

In some embodiments L is the peptide βA-PVGLIG-βA-C, wherein C (Cysteine) is optionally substituted and wherein βA is β-Alanine, wherein the peptide is connected to A via the peptide N terminus and to B via the peptide C terminus.

In some embodiments L is the peptide βA-PVGLIG-βA-C, wherein C (Cysteine) is optionally substituted and wherein βA is β-Alanine, wherein the peptide is connected to B via the peptide N terminus and to A via the peptide C terminus.

In some embodiments L is the peptide having the following structure :

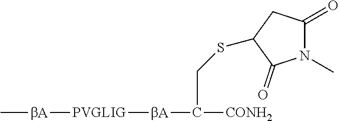

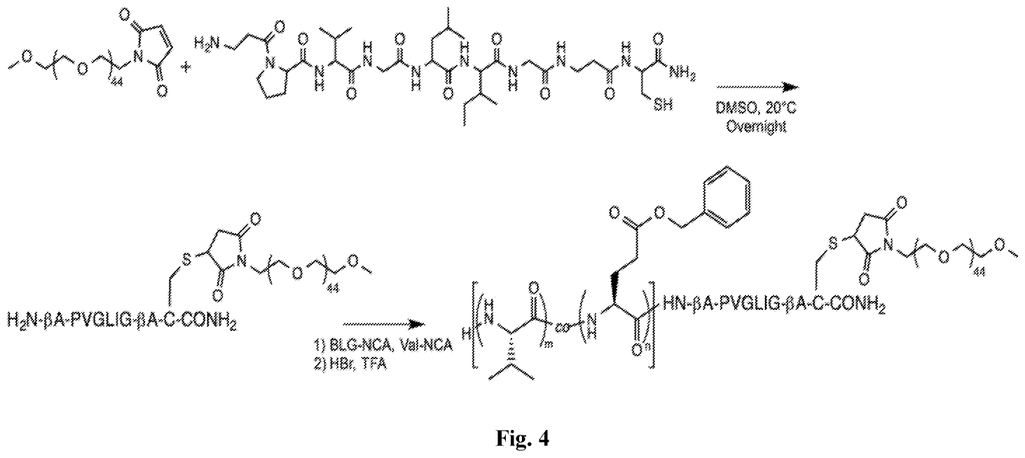

and wherein the peptide is connected to B via a peptide bond with the β-Alanine and to A via a N—C bond with the substituted cysteine moiety (See for example FIG. 4).

In some embodiments L is the peptide having the following structure:

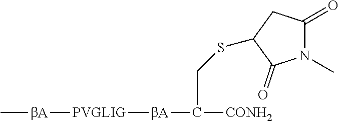

and wherein the peptide is connected to A via a peptide bond with the β-Alanine and to B via a N—C bond with the substituted cysteine moiety.

In some embodiments L is a peptide unit wherein the peptide unit is connected to A and/or B as herein exemplified.

In some embodiments L is selected from the peptide PVGLIG, the peptide βA-PVGLIG-βA-C (wherein C is optionally substituted) or the peptide having the following structure :

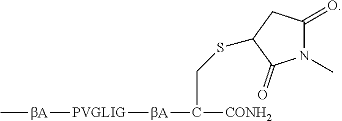

In some embodiments L is an enzymatically degradable linker segment.

In some embodiments L is an enzymatically degradable peptide.

In some embodiments according to the invention the nanoparticles e.g., nanocapsules are capable of releasing the active ingredient e.g., Dead Sea extract upon changes in pH and/or presence of enzymes encountered in the skin conditions.

Without wishing to be bound by theory, the release of the active ingredient may be due to change in the physico-chemical properties of the polymers of the amphipathic material. In some embodiments the change may be triggered by said changes in pH and/or presence of enzymes. For example, the superficial pH of healthy skin is around pH 5.5 to avoid bacterial contamination. However, in deeper layer and in the case of inflammation pH increases. The nanoparticles of the present invention are configured to react to this environment to release its payload e.g., Dead Sea salts and Minerals.

In some embodiments B in the amphipathic material comprises PGA. The polyglutamic acid (PGA) polymer block is pH-responsive due to the presence of carboxylic acid in each repeat unit. Then, at low pH glutamic acid (GA) units are sufficiently hydrophobic to adsorb/assemble at the oil water interphase due to the hydrophobic character of the carboxylic acid. However, at increasing pH the carboxylic acid deprotonates to form hydrophilic carboxylate anions which desorb form the interphase. This results in the destabilization of the nanoparticles and the release of its payload e.g., Dead Sea salts and Minerals.

In addition, the incorporation of a specific peptide group/s (L) in between both polymers of the amphipathic material i.e., A and B, induce release of the active ingredient in the presence of matrix metalloproteinase (MMP2 and MMP9) generated by some skin conditions e.g., photo damaging and irritant contact dermatitis. Further, in some embodiments wherein B is PGA, PGA would be degraded in the presence of Cathepsin B which is produced by the skin after UV exposure. Then, in the presence of those enzymes, the degradation of the polypeptide di-block copolymer will induce the release of the active ingredient. Such triggered release will increase the efficacy of the active ingredients as it will be released at the exact location where it is required.

In some embodiments the amphipathic material is selected to undergo dissociation and/or the linker L is selected to undergo dissociation from either polymer A and/or polymer B when in contact with an environment having one or more of:

(i) pH of between about 6.2 to 7.4;

(ii) presence of one or more of matrix metalloproteinase MMP2 and MMP9; and

(iii) presence of at least one Cathepsin B enzyme.

In some embodiments the amphipathic material is selected to undergo dissociation in the presence of at least one Cathepsin B enzyme.

In some embodiments the amphipathic material is selected to undergo dissociation at pH of between about 6.2 to 7.4.

In some embodiments the linker L is selected to undergo dissociation from either polymer A and/or polymer B when in contact with an environment having a presence of one or more of matrix metalloproteinase MMP2 and MMP9.

In some embodiments the nanoparticles according to the invention response to one or more of the following conditions and are characterized by having the detailed amphipathic material:

pH-responsive (wherein the amphipathic material is selected to undergo dissociation at pH of between about 6.2 to 7.4): PEG-PGA, i.e., A=PEG, L=absent (e.g., single bond, peptide bond) and B=PGA; or PGA-PEG i.e., A=PEG, L=absent (e.g., single bond, peptide bond) and B=PGA.
Enzymatic responsive (wherein the linker L is selected to undergo dissociation from either polymer A and/or polymer B when in contact with an environment having a presence of one or more of matrix metalloproteinase MMP2 and MMP9): PEG-PVGLIG-P(GA-VAL), i.e., A=PEG, L=PVGLIG and B=P(GA-VAL); or P(GA-VAL)-PVGLIG-PEG, i.e., A=PEG, L=PVGLIG and B=P(GA-VAL).
Dual responsive (wherein the amphipathic material is selected to undergo dissociation at pH of between about 6.2 to 7.4 and wherein the linker L is selected to undergo dissociation from either polymer A and/or polymer B when in contact with an environment having a presence of one or more of matrix metalloproteinase MMP2 and MMP9): PEG-PVGLIG-PGA i.e., A=PEG, L=PVGLIG and B=PGA; or P(GA-VAL)-PVGLIG-PEG, i.e., A=PEG, L=PVGLIG and B=P(GA-VAL).

In some embodiments the nanoparticles do not response to the above conditions and are characterized by having the detailed amphipathic material:

Non-responsive: (does not response to pH and Enzyme) PEG-P(GA-VAL) i.e., A=PEG, L=absent (e.g., single bond, peptide bond), and B=P(GA-VAL); or P(GA-VAL)-PEG i.e., A=PEG, L=absent (e.g., single bond, peptide bond), and B=P(GA-VAL).

Table 1 below illustrates some non-limiting examples of amphipathic materials of the forms A-L-B or B-L-A with the above properties:

TABLE 1

| | A | L | B |
|---|---|---|---|
| Non responsive | PEG | absent | P(GA-VAL) |
| pH responsive | PEG | absent | PGA |
| Enzyme responsive | PEG | PVGLIG | P(GA-VAL) |
| Dual responsive | PEG | PVGLIG | PGA |

In some embodiments the amphipathic material is of the form A-L-B or B-L-A, wherein:

A is an hydrophilic polymer (PEG)ₓ, wherein x is between about 4 to about 100 (at times between about 4 to about 120;

B is an hydrophobic polymer P(GA_{y}-Val_{z}), wherein y is between about 20 to about 100 (at times between about 20 to about 160) and z is 0 or at least about 30% of the value of y; and

L is a linker segment or a chemical bond associating between A and B.

In some embodiments x is 114.

In some embodiments y is 150 or 151 and z is 0.

In some embodiments y is 46 and z is 11.

In some embodiments x is 114, y is 150 or 151 and z is 0.

In some embodiments x is 114, y is 46 and z is 11.

In some embodiments L is an enzymatically degradable linker segment or a chemical bond associating between A and B.

In some embodiments L is an enzymatically degradable linker.

In some embodiments L is a peptide which may be an enzymatically degradable peptide.

In some embodiments L is a peptide selected from PVGLIG, βA-PVGLIG-βA-C (wherein C is optionally substituted) or a peptide having the following structure:

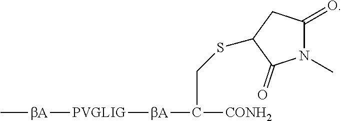

In some embodiments the amphipathic material is a copolymer of formula selected from the group consisting of:

(i) PEGₓ-PGA_{y} wherein x is between about 4 to about 120 (e.g., 114) and y is between about 20 to about 160 (e.g., 150, 151);

(ii) PGA_{y}-PVGLIG-PEGₓ wherein y is between about 20 to about 160 (e.g., 150, 151) and x is between about 4 to about 120 (e.g., 114); and

(iii) PEGₓ-PVGLIG-P(GA_{y}-Val_{z}) wherein x is between about 4 to about 120 (e.g., 114), y is between about 20 to about 160 (e.g., 46) and z is at least about 30% of the value of y (e.g., 11).

In some embodiments the amphipathic material is a copolymer of the formula PEGₓ-PGA_{y} wherein x is between about 4 to about 120 and y is between about 20 to about 160. In some embodiments said material is selected to undergo dissociation in environment having one or more of (i) pH of between about 6.2 to 7.4; and (ii) presence of at least one Cathepsin B enzyme.

In some embodiments the amphipathic material is a copolymer of the formula PGA_{y}-PVGLIG-PEGₓ wherein y is between about 20 to about 160 and x is between about 4 to about 120. In some embodiments said material is selected to undergo dissociation in environment having one or more of: (i) pH of between about 6.2 to 7.4; (ii) presence of one or more of matrix metalloproteinase MMP2 and MMP9; and (iii) presence of at least one Cathepsin B enzyme.

In some embodiments the amphipathic material is a copolymer of the formula PEGₓ-PVGLIG- PGA_{y} wherein x is between about 4 to about 120 and y is between about 20 to about 160. In some embodiments said material is selected to undergo dissociation in environment having one or more of: (i) pH of between about 6.2 to 7.4; (ii) presence of one or more of matrix metalloproteinase MMP2 and MMP9; and (iii) presence of at least one Cathepsin B enzyme.

In some embodiments the amphipathic material is a copolymer of the formula PEGₓ-PVGLIG-P(GA_{y}-Val_{z}) wherein x is between about 4 to about 120, y is between about 20 to about 160 and z is at least about 30% of the value of y. In some embodiments said amphipathic material is selected to undergo dissociation in environment having presence of one or more of matrix metalloproteinase MMP2 and MMP9.

In some embodiments the amphipathic material is a copolymer of the formula PEG₁₁₄-PGA₁₅₁.

In some embodiments the amphipathic material is a copolymer of the formula PGA₁₅₀-PVGLIG-PEG_{114.}

In some embodiments the amphipathic material is a copolymer of the formula PEG₁₁₄-PVGLIG-P(GA₄₆-Val₁₁).

An exemplary non-responsive amphipathic material is a copolymer of the formula PEG₁₁₄-b-P(GA₄₆-Val₁₁).

Exemplary applicable amphipathic materials, nanoparticles and methods of preparing same are disclosed in EP Application No. 17306354.6 and WO 2019/068936, the content of each is incorporated herein by reference.

Also, the emergence of controlled polymerization such as Atom Transfer Radical Polymerization (ATRP), Reversible Addition- Fragmentation chain-Transfer (RAFT), anionic polymerization, and Ring-Opening Polymerization (ROP) has allowed the architecture of synthetic polymer to be adjusted [5]. During the last decade, an immense number of academic works has been reported on the design of amphiphilic diblock copolymer which can self-assemble into micelles or vesicles depending on the ratio between the hydrophilic and hydrophobic block. More specifically, polypeptides diblock copolymers have attracted great interest due to their resemblance to naturally occurring proteins as well as over the control over intra- and intermolecular interactions.

As may be appreciated by a person versed in the art, the amphipathic materials of the present invention may be prepared by synthetic methods known in the art such as but not limited to the methods described in [5], [6] and [7], EP Application No. 17306354.6 and WO 2019/068936, the content of each is incorporated herein by reference.

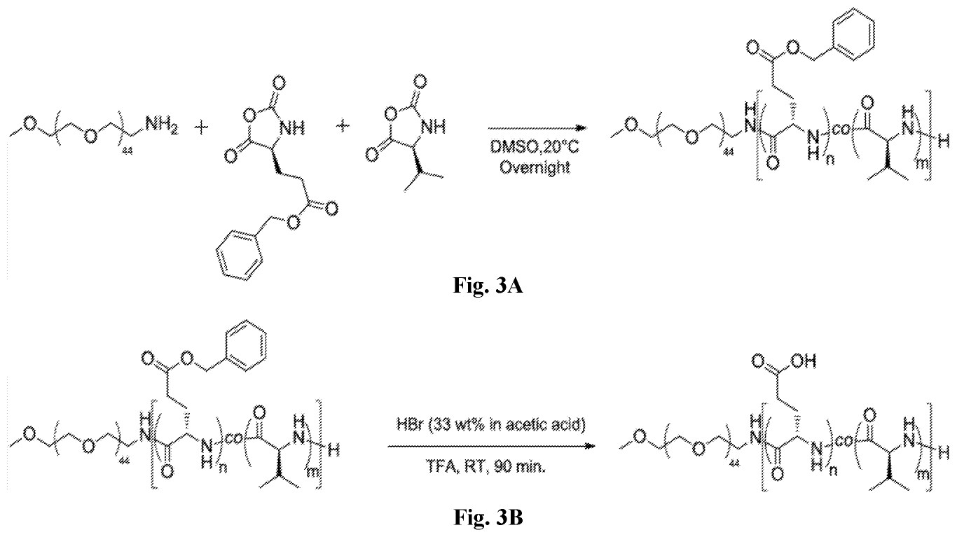

For example, FIG. 3A provides a schematic representation of the synthesis of poly(ethylene glycol)-block-poly(γ-benzyl-L-glutamate-co-L-valine) (PEG-b-P(BLG-co-Val). FIG. 3B provides a schematic presentation of the synthesis of poly(ethylene glycol)-block-poly(L-glutamate-co-L-valine) (PEG-b-P(GA-co-Val).

In FIGS. 3A-3B, in the PGA polymer of the amphipathic material of the invention, some GA units may be replaced with Val units. The number of GA units in the polymer is reflected in the integer “n” in these figures. The number of the Val units in the polymer is reflected in the integer “m” which at times may be at least 0.3 the value of “n”.

PGA polymerized via ROP is one interesting example of synthetic polypeptide due to the presence of carboxylic acid group in each glutamic acid [GA] unit which gave a pKa to this homopolymer located at pH 6.3. The presence of this group confers to the PGA homopolymer pH-responsive behavior where the polymer is hydrophobic at pH <6.3 due to the protonated carboxylic acid but becomes hydrophilic at pH >6.3 due to the deprotonated and highly hydrophilic carboxylate anions. Then, the preparation of diblock copolymer with PGA as one block allows the preparation of pH-responsive micelles and vesicles. Interestingly, PGA has been shown to be degraded in the presence of Cathepsin B enzymes.

Recently, specifically designed peptide sequence, namely PVGLIG, with the ability to be cleaved in the presence of matrix metalloproteinase (MMP2 and MMP9) has been conjugated to a polypeptide homopolymer, poly(trimethylene carbonate) [PTMC], via UV-initiated thiol-ene “click” chemistry [6]. The resulting diblock copolymer could self-assemble into polymeric vesicles which were completely degraded in the presence of MMP2 or MMP9. The same peptide conjugated with PTMC was used as macroinitiator to copolymerize a second block based on PGA. The resulting vesicle could encapsulate imipramine hydrochloride drug. Release of the drug was observed by change of pH and in the presence of MMP2 enzyme.

FIG. 4 provides a schematic presentation of the synthesis of an amphipatic polymer of PEG and PGA with the following linker

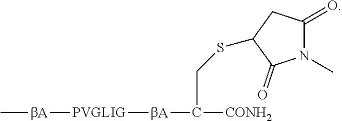

In FIG. 4, in the PGA polymer some GA units may be replaced with Val units. The number of GA units in the polymer is reflected in the integer “n” in the figure. The number of the Val units in the polymer is reflected in the integer “m” which at times may be at least 0.3 the value of “n”. Further, in FIG. 4, the PGA polymer is depicted with the protecting group of the carboxylic acid. The group is to be removed according to regular synthetic procedures know in the art.

In a further one of its aspects the present invention provides an emulsion comprising a plurality of the nanoparticles according to the invention.

In some embodiments in the amphipathic material of the form A-L-B or B-L-A a combination of A, L and B is configured as an amphipathic material for forming stable emulsions of one or more of a nanocarrier, a nanocapsule, a delivery system and an arraignment according to the invention.

In some embodiments stability of the nanoparticles and/or the emulsions thereof may be reflected in one or more of the stability of the particles size thereof and the colloidal stability. As may be appreciated by a person versed in the art, various known techniques may be used to determine the aforementioned stability.

In some embodiments the stability of the emulsions of the present invention may be reflected in absence of phase separation between the oil and aqueous phase.

The stability may be for a time period of several months e.g., at least 3 months (e.g., at room temperature). In some embodiments the emulsion is a water in oil (W/O) emulsion wherein the Dead Sea extract is present in the water dispersed phase and the continuous phase is an oil.

In some embodiments the emulsion is a water in oil (W/O) emulsion wherein the Dead Sea extract (e.g., DSW) constitutes the water dispersed phase and the continuous phase is an oil.

In some embodiments the oil is a vegetable oil.

In some embodiments the oil is a cosmetic oil.

In some embodiments the oil is a cosmetic oil selected from one or more of Octyl palmitate, Caprylic/Capric Triglyceride and Isodecyl Isononanoate.

In some embodiments the emulsion comprises a plurality of the nanocapsules according to the invention.

In some embodiments the emulsion comprises a plurality of the nanocapsules according to the invention wherein the Dead Sea extract is present or constitutes an aqueous core of the nanocapsule.

In a further one of its aspects the present invention provides an arraignment comprising a plurality of amphipathic materials around a core, wherein the core comprises at least one Dead Sea Extract, as an active ingredient, and wherein the plurality of amphipathic materials form a shell material substantially surrounding said core, said amphipathic materials each being of the form A-L-B or B-L-A, wherein A is an hydrophilic polymer, B is an hydrophobic polymer and L is a linker segment or a chemical bond associating between A and B.

In some embodiments the arraignment being of a nano-scale size.

In some embodiments the arraignment comprises a plurality of amphipathic materials as described herein above and below.

In a further one of its aspects the present invention provides delivery system for the delivery of at least one Dead Sea Extract to at least one skin region, wherein the system comprises a nanoparticle comprising at least one Dead Sea Extract, as an active ingredient, and at least one amphipathic material of the form A-L-B or B-L-A as described herein above and below.

In some embodiments the skin region may be the stratum corneum skin layer.

In some embodiments the skin region may be the upper epidermal layers of the skin.

In some embodiments the skin region may be an inflammable region.

In some embodiments the delivery system comprises a nanocapsule comprising a core and a shell material, wherein the core comprises at least one Dead Sea Extract, as an active ingredient, and the shell material comprises at least one amphipathic material of the form A-L-B or B-L-A as described herein above and below.

In some embodiments the delivery system comprises the nanoparticle according to the invention (e.g., a nanocapsule or a nanocarrier) and/or the emulsion according to the invention.

In another one of its aspects the present invention provides a composition comprising the nanoparticle according to the invention.

In some embodiments the composition may be a skin-care composition (or formulation) and/or a pharmaceutical composition (or formulation).

The nanoparticles and/or emulsions and/or compositions and/or formulations and/or delivery system and/or the arraignment according to the present invention have therapeutic uses as herein disclosed.

In another one of its aspects the present invention provides the nanoparticles and/or delivery system and/or arraignment according to the invention for use in the preparation of skin-care and/or pharmaceutical formulations.

Non limiting examples of therapeutic indications of the nanoparticles and/or emulsions and/or compositions and/or formulations and/or delivery system and/or the arraignment according to the present invention are one or more of the following:
Reducing and/or preventing and/or treating contact dermatitis e.g., irritant contact dermatitis (the most common type of ICD).
Reducing and/or preventing and/or treating allergic contact dermatitis [e.g., that results when repeated exposure to an allergen (sensitization) triggers an immune response that inflames the skin].
Reducing and/or preventing and/or treating skin photo-aging (e.g., that relates to the effect on the skin exposed to UVA and UVB which damage dramatically the skin resulting in premature wrinkling, sagging, a leathery texture and hyperpigmentation and may eventually lead to skin cancer).
Reducing and/or preventing and/or treating skin pigmentation disorders such as age spots, freckles and post-inflammation spots (skin pigmentation, can be caused by sun damage, inflammation, or other skin injuries, including those related to acne vulgaris, Hyperpigmentation, or brown spots).

In some embodiments of the invention the active Dead Sea ingredient may be present in the nanoparticles according to the invention in combination with at least one another active material which at times may provide a synergistic effect.

A non-limiting example of a combination of active ingredients according to the present invention is: a combination of Vitamin C, Dead Sea extract, at least one anti-oxidant and Hyaluronic acid (which may be encapsulated in an hydrophilic core of a nanocapsule according to the present invention). Such combinations may be used in the treatment of one or more of ACD, ICD skin photo-damage and skin pigmentation.

A further non limiting example of a combination of active ingredients according to the present invention is: a combination of Vitamin D, Dead Sea extract, at least one retinoid and Hyaluronic acid (which may be encapsulated in an hydrophilic core of a nanocapsule according to the present invention). Such combinations may be used in the treatment of damaged skin e.g., by promoting skin regeneration.

A further non limiting example of a combination of active ingredients according to the present invention is: a combination of Vitamin C, Dead Sea extract, Curcumin and nitorxide.

A further non limiting example of a combination of active ingredients according to the present invention is: a combination of at least one antioxidant e.g., Vitamin C and Dead Sea extract. Such combinations may be used in the treatment of skin pigmentation.

A further non limiting example of a combination of active ingredients according to the present invention is: a combination of at least one photosensitive e.g., Vitamin D and Dead Sea extract.

A further non limiting example of a combination of active ingredients according to the present invention is: a combination of at least one polymeric compound e.g., hyaluronic acid and Dead Sea extract.

A further non limiting example of a combination of active ingredients according to the present invention is: a combination of at least one Niacinamide (a soluble form of vitamin B) and Dead Sea extract. Such combinations may be used in the treatment of skin pigmentation.

A further non limiting example of a combination of active ingredients according to the present invention is: a combination of at least one Nitroxide and Dead Sea extract. Such combinations may be used in the treatment of skin pigmentation.

A further non limiting example of a combination of active ingredients according to the present invention is: a combination of at least one Dipotassium glycyrrhizinate and Dead Sea extract. Such combinations may be used in the treatment of ICD and/or ACD.

A further non limiting example of a combination of active ingredients according to the present invention is: a combination of at least one Curcumin and Dead Sea extract. Such combinations may be used in the treatment of ICD and/or ACD.

In some embodiments the Dead Sea extract is present in the nanoparticle in combination with one or more of:

a) Vitamin C, at least one anti-oxidant and Hyaluronic acid;

b) Vitamin D, at least one retinoid and Hyaluronic acid;

c) Vitamin C, Curcumin and nitorxide;

d) Vitamin C;

e) Vitamin D;

f) Hyaluronic acid;

g) at least one Niacinamide;

h) at least one Nitroxides;

i) at least one Dipotassium glycyrrhizinate; and

j) at least one Curcumin.

As used herein, the expression “active ingredient” refers to the ability of the ingredient to exert a protective/preventive skin-care/therapeutic effect, as disclosed herein.

As used herein the term “Dead Sea extract” refers to one or more natural material, in the form of a single material (e.g., inorganic, organic, salt, etc.) or a mixture of natural materials obtained from the waters of the Dead Sea and/or the mud surrounding the Dead Sea and/or the soil bed of the Dead Sea.

In some embodiments the Dead Sea extract is a mixture of natural materials (e.g., salts, minerals) obtained from the waters of the Dead Sea and/or the mud surrounding the Dead Sea and/or the soil bed of the Dead Sea.

In some embodiments the Dead Sea extract is a mixture of natural materials (e.g., salts, minerals) obtained from the waters of the Dead Sea.

In some embodiments, the Dead Sea extract is the Dead Sea water.

As used herein the term “Dead Sea water” (herein abbreviated DSW) refers to the saline waters obtained from the Dead Sea (Israel or Jordan) region or an aqueous solution prepared by dissolving Dead Sea minerals in an aqueous medium. The term also encompasses aqueous solutions which simulate such natural solution, namely having at least one parameter substantially identical to that measured for the natural DSW, said parameter being at least one of salt content, at least one of mineral content, salt concentration, concentration of a particular cation or anion, ratio of divalent cations to monovalent cations, TDS (Total Dissolved Salt, w/v), soluble natural substances, and other parameters known to define or characterize natural DSW.

In some embodiments the Dead Sea extract is an aqueous solution having salt and mineral content substantially identical to that measured for the natural DSW.

In some embodiments the Dead Sea extract is an aqueous solution having substantially the same salt (a hypersaline concentration) and mineral content as that of the Dead Sea water.

In some embodiments, the Dead Sea extract is the Dead Sea water which may be obtained directly from the Dead Sea filtered water substantially having the same salt content (a hypersaline concentration) as that of the unfiltered Dead Sea water, or Dead Sea water treated by any one or more of various other methods employed to e.g., remove organic matter and residual contaminants therefrom.

In some embodiments, the Dead Sea extract is an aqueous solution simulating the content of DSW i.e., having substantially identical content as that of DSW.

In some embodiments, the Dead Sea extract is an aqueous solution having substantially identical salts content, minerals content, salts concentration and mineral concentrations as that of DSW.

In some embodiments, the Dead Sea extract is an aqueous solution having substantially identical salts content, minerals content, salts concentration, minerals concentrations, concentration of a particular cation or anion, ratio of divalent cations to monovalent cations, TDS, soluble natural substances and other parameters known to define or characterize natural DSW.

In some embodiments, the Dead Sea extract is an aqueous solution simulating the salt content (a hypersaline concentration) of DSW i.e., having salt content substantially identical to that of DSW.

In some embodiments, the Dead Sea extract is an aqueous solution simulating the mineral content of DSW i.e., having mineral content substantially identical to that of DSW.

In some embodiments, the Dead Sea extract is an aqueous solution simulating the salt content (a hypersaline concentration) and the mineral content of DSW i.e., having salt content substantially identical to that of DSW and mineral content substantially identical to that of DSW.

In some embodiments, the Dead Sea water having:

1. a specific density of 1.25-1.35 g/ml,

2. pH=4.6-5.6 (at 25° C.), and/or

3. less than 100 cfu/g of non-pathogenic microbes.

The Dead Sea water having the above physical characteristics is a concentrated extract of Dead Sea water comprising (among other metal salt ions) Ca⁺², Mg⁺², Na⁺ and K⁺ and high concentrations of anions such as Cl⁻ and Br⁻.

In some embodiments, the DSW is a clear colorless viscous liquid (at 25° C.).

In some embodiments, the concentrations of these ions are, as assessed by a water analysis carried out by the Geological Survey of Israel:
Calcium (Ca⁺²): 35,000-40,000 mg/L
Chloride (Cl⁻): 320,000-370,000 mg/L
Magnesium (Mg⁺²): 92,000-95,000 mg/L
Sodium (Na⁺): 1800-3200 mg/L
Potassium (K⁺): 2,500 mg/L, and
Bromide (Br⁻): 10,000-12,000 mg/L.
   Other minerals may also exist in the waters.

In some embodiments, the Dead Sea Water comprises:
Calcium (Ca⁺²): 35,000-40,000 mg/L
Chloride (Cl⁻): 320,000-370,000 mg/L
Magnesium (Mg⁺²): 92,000-95,000 mg/L
Sodium (Na⁺): 2400-3200 mg/L
Potassium (K⁺): 2,500 mg/L, and
Bromide (Br⁻): 10,000-12,000 mg/L.
   Other minerals may also exist in the waters.

In some embodiments, the Dead Sea Water comprises:
Calcium (Ca²⁺): 5,000-10,000 mg/L
Chloride (Cl⁻0: 315,000-360,000 mg/L
Magnesium (Mg⁺²): 100,000-150,000 mg/L
Sodium (Na⁺): 1800-2200 mg/L
Potassium (K⁺): 1,000-2,000 mg/L, and
Bromide (Br⁻): 5,000-10,000 mg/L.
   Other minerals may also exist in the waters.

In some further embodiments, the Dead Sea Water comprises:
Calcium (Ca⁺²) 34,000-40,000 mg/L
Chloride (Cl⁻) 320,000-370,000 mg/ L
Magnesium (Mg⁺²) 90,000-95,000 mg/L
Potassium (K⁺) 1,300-2,200 mg/L
Sodium (Na⁺) 1,500-2,800 mg/L
Bromide (Br⁻) 11,000-15,000 mg/L.
   Other minerals may also exist in the waters.

In some embodiments, the Dead Sea Water comprises:
Calcium (Ca⁺²): 38,000 mg/L
Chloride (Cl⁻): 345,000 mg/L
Magnesium (Mg⁺²): 92,500 mg/L
Sodium (Na⁺): 2000 mg/L
Strontium (Sr⁺²): 800 mg/L
Potassium (K⁺): 1,400 mg/L, and
Bromide (Br⁻): 11,500 mg/L.
   Other minerals may also exist in the waters.

In some embodiments, the Dead Sea Water comprises:
Calcium (Ca⁺²): 38,000 mg/L
Chloride (Cl⁻): 345,000 mg/L
Magnesium (Mg⁺²): 92,500 mg/L
Sodium (Na⁺): 2000 mg/L
Strontium (Sr⁺²): 800 mg/L
Potassium (K⁺): 1,400 mg/L, and
Bromide (Br⁻): 11,500 mg/L.
   Other minerals may also exist in the waters.

In some embodiments, the DSW is natural DSW which has undergone pre-treatment, e.g., having been concentrated by allowing water to evaporate, for example through solar evaporation, thereafter reconstituted to afford a solution.

In some embodiments the Dead Sea extract is Dead Sea Water preparation commercially available as “Maris Sal” or “Maris Aqua” (AHAVA, Israel) referred to herein below also as “Osmoter”.

In some embodiments the Dead Sea extract is Dead Sea mud.

The Dead Sea extract is typically an active fraction having by itself at least one attribute which may be enhanced while entrapped in the nanoparticles according to the present invention.

In some embodiments the concentration of the Dead Sea extract (e.g., Dead Sea water) in the nanoparticles of the invention (and/or in the compositions/formulations of the invention) is at least about 0.01% (w/w). At times it is between about 0.01% to about 7.5%. At times is it about 0.1%, 0.2%, 0.3%, 0.4%, 0.5%, 0.6%, 0.7%. 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4% and 2.5%. At times it is about 1%, 2%, 3%, 4%, 5%, 6%, 7%. At times it is about 7.1%. Any value which is between any one of the above values is within the scope of the present disclosure.

In some embodiments the concentration of the Dead Sea extract (e.g., Dead Sea water) in the core of the nanocapsule of the invention is at least about 0.01% (w/w). At times it is between about 0.01% to about 7.5%. At times is it about 0.1%, 0.2%, 0.3%, 0.4%, 0.5%, 0.6%, 0.7%. 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4% and 2.5%. At times it is about 1%, 2%, 3%, 4%, 5%, 6%, 7%. At times it is about 7.1%. Any value which is between any one of the above values is within the scope of the present disclosure.

In some embodiments, the nanoparticles/delivery system of the invention are comprised within a composition which may be a cosmetic composition and/or a pharmaceutical composition for topical application.

The compositions of the present invention may be made into a wide variety of product forms suitable for, e.g., topical administration onto the skin of a subject. Non-limiting examples are a lotion, an ointment, a gel, a mask, a toner, an essence, a shampoo, a moisturizer, a sunscreen, a cream, a stick, a spray, an aerosol, foam, a paste, a mousse and a variety of cosmetics or skin-care formulations including solid, semi-solid, or a liquid make-up such as foundations, eye make-up, etc.

Thus, in another one of its aspects the present invention provides one or more of a lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, and an eye make-up comprising the nanoparticles/compositions/formulations/delivery system/emulsions according to the invention.

In some embodiments the liquid may be applied onto the skin as a moisturizer.

In some embodiments, the composition of the invention is formulated as a lotion.

In some embodiments, the composition of the invention is formulated as an emulsion.

In some embodiments, the composition of the invention is formulated as a facial formulation.

In some embodiments, the composition of the invention is formulated as a body formulation.

In some embodiments, the composition of the invention is formulated as a leave on formulation.

In some embodiments, the composition of the invention is formulated as rinse off formulation.

As used herein, a “leave on” (in contrary to “rinse off”) composition/formulation refers to a composition/formulation that may be in prolonged contact with the skin and can be applied to a skin region without the need to remove it from the skin (e.g., by wiping or rinsing it off) in any way.

In some embodiments, the leave-on composition/formulation may be adapted to be applied to a skin region and to be left on the skin for a time sufficient to achieve an end result.

The compositions according to the invention (cosmetic or therapeutic) may comprise at least one dermatological, cosmetically or pharmaceutically acceptable additive selected amongst inert and effect-inducing additives. In some embodiments, the inert additive is selected from a diluent, a preservative, an abrasive, an anti-caking agent, an antistatic agent, a binder, a buffer, a dispersant, an emollient, an emulsifier, a co-emulsifiers, a fibrous material, a film forming agent, a fixative, a foaming agent, a foam stabilizer, a foam booster, a gallant, a lubricant, a moisture barrier agent, an opacifier (e.g., styrene/acrylamide copolymer), a plasticizer, a preservative, a propellant, a stabilizer, a surfactant, a suspending agent, a thickener, a wetting agent, and a liquefier.

In some embodiments, the at least one inert additive is a smoothness enhancer ingredient, such as silica.

In some embodiments, each of the at least one dermatological, cosmetically or pharmaceutically acceptable additives may constitute between about 0.05 to 15% of the total weight of the formulation. In some embodiments, the at least one additive constitutes between 0.05% and 10% or between 0.05% and 8%, or between 0.05% and 7%, or between 0.05% and 6%, or between 0.05% and 5% of the total weight of the formulation.

In some embodiments, the at least one inert additive is a diluent being selected from water, Bisabolol, propane diol, propylene glycol, butylene glycol, glycerin, safflower oil and mixtures thereof.

In some embodiments, the at least one inert additive is a preservative being selected from one or more of methylparaben, methyldibromo glutaronitrile, phenethyl alcohol, glyceryl caprilate, propylparaben, methylisothiazolinone, decylene glycol, dehydroacetic acid, phenoxyethanol, benzoic acid, 2-methyl-2H-isothiazoline-3-one, polyethylene glycol monococoate, polyethylene glycol dicocoate, polyethylene Glycol, iodopropynyl butylcarbamate, 1.2-hexanediol, caprylyl glycol, imidazolidinyl urea, DMDM Hydantoin, Ipbc, MIT, 2,3-bronopol.

In further embodiments, the inert additive is an emulsifier being selected from one or more of cetyl hydroxyethylcellulose, cetyl alcohol, ceteth-20 (a polyethylene glycol derivative of cetyl alcohol), cetearyl olivate, cetyl palmitate, sorbitan olivate, sorbitan palmitate, stearates, steareth-20 (polyethylene glycol ethers of stearic acid- octadecyl polyoxyethylene ether), steareth-25.

In some embodiments, the stearate is selected from PEG-40 stearate, glyceryl steatrate, sorbitan tristearate, stearyl alcohol and mixtures thereof.

In some embodiments, the stearate is glyceryl stearate.

In still other embodiments, the inert additive is an emollient, being selected from vegetable and animal fats and oils such as castor oil, hydrogenated castor oil, cocoa butter, safflower oil, cottonseed oil, corn oil, olive oil, cod liver oil, almond oil, avocado oil, palm oil, sesame oil, squalene, phytosqalene, kikui oil, Chamomilla recutita (matricaria) flower oil, hypericum perforatum oil, soybean oil and Vitis vinifera (grape) seed oil; acetoglyceride esters, such as acetylated monoglycerides; alkyl esters of fatty acids having 10 to 24 carbon atoms which include, but are not limited to, methyl, isopropyl, and butyl esters of fatty acids such as hexyl laurate, isohexyl laurate, ethylhexyl palmitate, isohexyl palmitate, isopropyl palmitate, octyl palmitate, decyloleate, isodecyl oleate, hexadecyl stearate decyl stearate, isopropyl isostearate, diisopropyl adipate, diisohexyl adipate, dihexyldecyl adipate, diisopropyl sebacate, lauryl lactate, myristyl lactate, and cetyl lactate; alkenyl esters of fatty acids having 10 to 20 carbon atoms such as oleyl myristate, oleyl stearate, and oleyl oleate; fatty acids having 10 to 20 carbon atoms such as pelargonic, lauric, myristic, palmitic, stearic, isostearic, hydroxystearic, oleic, linoleic, ricinoleic, arachidic, behenic, and erucic acids; fatty alcohols having 10 to 20 carbon atoms such as lauryl, myristyl, cetyl, hexadecyl, stearyl, isostearyl, hydroxystearyl, oleyl, ricinoleyl, behenyl, erucyl, and 2-octyl dodecanyl alcohols; fatty alcohol ethers such as propoxylated fatty alcohols of 10 to 20 carbon atoms which include, but are not limited to, lauryl, cetyl, stearyl, isostearyl, oleyl, and cholesterol alcohols, having attached thereto from 1 to 50 propylene oxide groups; lanolin and lanolin derivatives such as lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, bydrogenolysis of lanolin, ethoxylated sorbitol lanolin, and liquid and semisolid lanolin absorption bases; polyhydric alcohol esters such as ethylene glycol mono and di-fatty acid esters, diethylene glycol mono-and di-fatty acid esters, polyethylene glycol (200-6000) mono-and di-fatty acid esters, propylene glycol mono-and di-fatty acid esters, polypropylene glycol 2000 monooleate, polypropylene glycol 2000 monostearate, glyceryl mono-and di-fatty acid esters, polyglycerol polyfatty esters, ethoxylated glyceryl monostearate, 1,2-butylene glycol monostearate, 1,2-butylene glycol distearate, polyoxyethylene polyol fatty acid ester, sorbitan fatty acid esters, and polyoxyethylene sorbitan fatty acid esters; Wax esters such as beeswax, spermaceti, myristyl myristate, stearyl stearate; forming a mixture of ether esters; vegetable waxes including, but not limited to, carnauba and candelilla waxes; surface active silicone derivatives such as cyclopentasiloxane PEG/PPG-18/18 dimethicone, dimethicone, dimethicone crosspolymer, cyclomethicone, cyclomethicone&dimethiconol; caprylic/capric triglyceride; and cholesterol fatty acid esters and any mixtures thereof.

In some embodiments the oil is a vegetable oil.

In some embodiments the oil is a cosmetic oil

In some embodiments the oil is a cosmetic oil selected from Octyl palmitate, Caprylic/Capric Triglyceride or Isodecyl Isononanoate.

In some embodiments, each of the at least one inert additive may constitute between about 0.05 to 15% of the total weight of the composition/formulation. In some embodiments, the at least one inert additive constitutes between 0.05% and 10% or between 0.05% and 8%, or between 0.05% and 7%, or between 0.05% and 6%, or between 0.05% and 5% of the total weight of the composition/formulation.

In some embodiments, the effect-inducing additive is selected from an anti-acne agent, an anti-aging agent, an antibacterial agent, an anti-cellulites agent, an antidandruff agent, an antifungal agent, an anti-inflammatory agent, an anti-irritation agent (e.g., allantoin, Aloe Barbadensis leaf juice), an antimicrobial agent, an antioxidant (e.g., butylated hydroxyanisole, propyl gallate, an antiperspirant agent, an antiseptic agent, a cell stimulant, a cleansing agent, a conditioner, a deodorant, a fragrance ingredient (e.g., perfume, limonene), a depilatory, a detergent, an enzyme, an essential oil, an exfoliant, a fungicide, a glosser, hair conditioner (hair conditioner agent), hair set resin, hair sheen agent, hair waving agent, a humectants (e.g., Erythritol, Homarine HCl, Ceratonia siliqua (carob bean) gum), a moisturizer (e.g., sodium hyaluronate), an ointment base, a perfume, a protein, a skin calming agent, a skin cleanser, a skin conditioner (skin conditioning agent), a skin healing agent, a skin lightening agent, a skin protectant, a skin smoothing agent, a skin softening agent, a skin soothing agent, a sunscreen agent, a tanning accelerator, vitamins, a colorant, and a flavoring agent.

In some embodiments, the at least one additive is a sunscreen, such as Ethyl hexyl methoxycinnamate or titanium dioxide.

In some embodiments, each of the at least one effect-inducing additive may constitute between about 0.05 to 15% of the total weight of the formulation. In some embodiments, the at least one inert additive constitutes between 0.05% and 10% or between 0.05% and 8%, or between 0.05% and 7%, or between 0.05% and 6%, or between 0.05% and 5% of the total weight of the composition/formulation.

The cosmetic or pharmaceutical compositions/formulations of the invention may also comprise pharmaceutical actives useful in the form of a chemical substance, material or compound, e.g., suitable for topical administration, to induce a desired local or systemic effect. Non-limiting examples of such actives are an antibiotic, an antiviral agent, an analgesic (e.g. ibuprofen, acetyl salicylic acid, naproxen, and the like), an antihistamine, an anti-inflammatory agent, an antipruritic, an antipyretic, an anesthetic agent, a diagnostic agent, a hormone, an antifungal agent, an antimicrobial agent, a cutaneous growth enhancer, a pigment modulator, an antiproliferative, an antipsoriatic, a retinoid, an anti-acne medicament (e.g. benzoyl peroxide, sulfur, and the like), an antineoplastic agent, a phototherapeutic agent, a keratolys (e.g. resorcinol, salicylic acid, and the like) and mixtures thereof.

Application of a composition/formulations of the invention onto the skin of a subject, for cosmetic/skin-care or therapeutic purposes may be in a single dose, in multiple doses, in a continuous or intermittent manner, depending, for example, upon the subject's physiological condition, whether the purpose of the administration is cosmetic or therapeutic/prophylactic and other factors known to the medical practitioner. The application of a composition/formulations of the invention may be essentially continuous over a pre-selected period of time or may be in a series of spaced doses.

The compositions/formulations of the invention, being substantially for topical use, may be a skin-care formulation or a therapeutic formulation.

In some embodiments, the compositions/formulations of the invention are skin-care or dermo-pharmaceutical compositions/formulations (including, e.g., toiletries, health and beauty aids and cosmeceuticals) used for cosmetic and personal skin-care applications.

The term “cosmetic composition/formulation” or “skin care composition/formulation” relates to a composition of the invention that can be used for cosmetic purposes, purposes of hygiene or skin-care or as a basis for delivery of one or more pharmaceutical ingredients. It is also possible that these compositions are used for two or more of these same purposes at one time. For example, a formulation may be used as a personal care product and at the same time have pharmacological properties.

In some embodiments, the cosmetic compositions/formulations are for promoting bodily attractiveness, cover or mask the physical manifestations of a disorder or disease, modulate or alleviate wrinkling, unevenness and dryness in the skin of a mammal. The compositions/formulations may additionally regulate skin condition and signs of skin aging (all perceptible manifestations as well as any other macro or micro effects) by regulating visible and/or tactile discontinuities in skin texture, including fine lines, wrinkles, enlarged pores, roughness and other skin texture discontinuities associated with aged skin with reduced irritation and dryness.

It is noted that the embodiments detailed herein in connection with the compositions and/or formulations of the invention (e.g., with respect to dermatological, cosmetically or pharmaceutically acceptable additive such as inert and effect-inducing additives) may also be applicable to the nanoparticles (nanocarriers/nocapsules), delivery systems, arraignments and emulsions of the invention.

In another one of its aspect the present invention provides a composition (formulation) according to the invention for one or more of protecting and/or improving the state of the skin, and preventing and/or treating imperfections of the skin of a subject in need thereof.

In some embodiments the state of the skin and/or the imperfections of the skin may be associated with skin environment having one or more of (i) pH of between about 6.2 to 7.4; (ii) presence of one or more of matrix metalloproteinase MMP2 and MMP9; and (iii) presence of at least one Cathepsin B enzyme.

In a further one of its aspects the present invention provides the nanoparticles/compositions/formulations/delivery system/emulsion according to the invention for one or more of protecting and/or improving the state of the skin, and preventing and/or treating imperfections of the skin of a subject.

Yet, in a further one of its aspects the present invention provides the nanoparticles/compositions/formulations/delivery system/arraignment/emulsions according to the invention for treating or preventing at least one disease or disorder of the skin.

In a further one of its aspect the present invention provides the use of the nanoparticle/delivery system/arraignment according to the invention for the preparation of a pharmaceutical composition for treating or preventing a disease or disorder of the skin.

According to another one of its aspect the present invention provides a composition (formulation) according to the invention for use in a method of one or more of protecting and/or improving the state of the skin, and preventing and/or treating imperfections of the skin of a subject in need thereof.

The invention further provides according to one of its aspect a method of one or more of protecting and/or improving the state of the skin, and preventing and/or treating imperfections of the skin of a subject in need thereof, said method comprising topically administering a composition according to the invention onto the skin of said subject.

The invention further provides according to another one of its aspect a method for treating or preventing a disease or disorder of the skin of a subject, the method comprises administering to a subject in need thereof nanoparticles and/or compositions and/or formulations and/or delivery system and/or arraignment according to the invention.

In some embodiments the disease or disorder may be associated with skin environment having one or more of (i) pH of between about 6.2 to 7.4; (ii) presence of one or more of matrix metalloproteinase MMP2 and MMP9; and (iii) presence of at least one Cathepsin B enzyme.

In some embodiments the disease or disorder are selected from irritant contact dermatitis (ICD), allergic contact dermatitis (ACD), skin photo-aging and skin pigmentation.

In some embodiments the disease/disorder is ICD.

In some embodiments the disease/disorder is ACD.

In some embodiment the disease/disorder is skin photo-aging.

In some embodiments the disease/disorder is skin pigmentation.

In some embodiments the skin condition may be associated with environment having one or more of (i) pH of between about 6.2 to 7.4; (ii) presence of one or more of matrix metalloproteinase MMP2 and MMP9; and (iii) presence of at least one Cathepsin B enzyme.

In some embodiments, the methods and/or nanoparticles and/or compositions and/or formulations and/or delivery system and/or arraignment according to the invention are used for treating rings under the eye, symptoms of aging, protecting the skin, increasing the detoxification of xenobiotics, intervening on pigmentation level, inhibiting melanogenesis, stimulating the detoxification systems, stimulating hair and body hair growth, modulating DHT levels, intervening on adipocytes, and promoting lipolysis.

In some embodiments, the methods and/or compositions and/or formulations and/or delivery system of the invention are used for one or more of rejuvenating the skin, improving the state of the skin, preventing and/or treating imperfections of the skin of a subject, protecting and/or improving the state of the skin, the treatment or prevention of at least one disease or disorder (e.g., of the skin).

In some embodiments, the disease or disorder of the skin is related to sun exposure.

In some embodiments, the disease or disorder of the skin is a secondary condition, e.g., inflammation, being related to an existing condition.

In some embodiments, the disease or disorder of the skin is skin irritation which may be related to an existing condition.

In further embodiments, the disease or disorder of the skin are age-related.

Non-limiting examples of such diseases or disorders of the skin are wounds, acne, psoriasis, atopic skin, diabetic skin, dermatitis, eczema, xerotic, dry skin, and chaffed skin.

In some embodiments the compositions and/or formulations and/or methods and/or delivery system and/or arraignment of the present invention may be used for wound healing.

In some embodiments the compositions and/or formulations and/or methods and/or delivery system and/or arraignment of the present invention may be used for the treatment of infection of the skin.

The term “topical” as used herein above and below refers to the application of a composition and/or formulation and/or emulsion and/or delivery system and/or assembly according to the invention directly onto at least a portion of a subject's skin (human's or non-human's skin) so as to achieve a desired effect, e.g., cosmetic or therapeutic effect, at the site of application. In some embodiments, the desired effect is achieved at the site of application without inducing one or more systemic effects. In other embodiments, the composition and/or formulation and/or emulsion and/or delivery system and/or assembly and/or nanoparticles of the invention induces at least a partial systemic effect which contributes to the induction of at least one desired effect.

The term “skin” as used herein above and below refers to any part of the human or animal skin, including the whole surface thereof, hair and nails.

The term “treatment” as used herein above and below refers to administration (e.g., topical) of an effective amount of a composition and/or formulation and/or emulsion and/or delivery system and/or nanoparticle and/or assembly of the present invention effective to ameliorate undesired symptoms associated with a disease/disorder (e.g., skin disease), to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

In some embodiments the disease and/or disorder is a non-medical condition e.g., associated with normal skin conditions.

In some embodiments the disease and/or disorder is a medical condition e.g., associate with pathological skin conditions.

The “effective amount”, whether a therapeutically or cosmetically effective amount for purposes disclosed herein, is determined by such considerations as may be known in the art. The amount must be effective to achieve one or more of the above desired therapeutic or cosmetic effects, depending, inter alia, on the type and severity of the disease/condition to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile, a variety of pharmacological parameters such as half-life on the skin, on undesired side effects, if any, on factors such as age and gender, etc.

As used herein above and below the term “about” refers to ±10% of the indicated value.

In a further one of its aspects the present invention provides a method for the encapsulation of at least one Dead Sea Extract, the method comprising:

providing a water solution comprising at least one Dead Sea Extract and optionally an amphipathic material;
providing an oil solution comprising at least one oil and optionally an amphipathic material;
provided that one of said water solution or oil solution comprises the amphipathic material and being at a pH of between about 4.0 to about 6.0, the amphipathic material being substantially as disclosed herein above and below; and

applying means to disperse the aqueous solution into the oil solution to thereby obtain a nanocapsule comprising a core and a shell, wherein said core comprises the at least one Dead Sea Extract and wherein said shell comprises the amphipathic material which is substantially continuously assembled around the core.

Yet, in a further one of its aspects the present invention provides a method for the encapsulation of at least one Dead Sea Extract, the method comprising:

providing a water solution comprising at least one Dead Sea Extract and an amphipathic material of the form A-L-B or B-L-A, wherein A is an hydrophilic polymer, B is an hydrophobic polymer and L is a linker segment or a chemical bond associating between A and B, wherein said aqueous solution is at a pH of between about 4.0 (inclusive) to about 6.0 (inclusive);

providing an oil solution comprising at least one oil;

applying means to disperse the aqueous solution into the oil solution to thereby obtain a nanocapsule comprising a core and a shell, wherein said core comprises the at least one Dead Sea Extract and wherein said shell comprises the amphipathic material which is substantially continuously assembled around the core.

In some embodiments the aqueous solution is at a pH of between about 4.0 (inclusive) to about 6.0 (inclusive). Any value which is between any one of pH 4.0 and pH 6.0 is within the scope of the present disclosure.

In some embodiments the aqueous solution is at a pH of 4.0, 4.1, 4.2, 4.3, 4.4, 4.5, 4.6, 4.7, 4.8, 4.9, 5.0, 5.1, 5.2, 5.2, 5.3, 5.4, 5.5, 5.6, 5.7, 5.8, 5.9 or 6.0. In some embodiments the aqueous solution is at a pH of about 5.5.

In some embodiments the water solution may optionally further comprise at least one another material such as a carrier, diluent, an excipient, a surfactant and the like, or any combination thereof.

In some embodiments the at least one another material may be a non-active and/or an active material.

In some embodiments the water solution may optionally further comprise at least one another active material e.g. as herein described.

In some embodiments the water solution may optionally further comprise at least one steric stabilizing agent.

In some embodiments the core of the nanocapsule comprises a single droplet of water comprising the at least one Dead Sea Extract.

In some embodiments the core of the nanocapsule comprises a single droplet of Dead Sea water.

In some embodiments the core of the nanocapsule may optionally further comprise at least one another material such as a carrier, diluent, an excipient, a surfactant and the like, or any combination thereof.

In some embodiments the at least one another material may be a non-active and/or an active material.

In some embodiments the core of the nanocapsule may optionally further comprise at least one another active material e.g. as herein described.

In some embodiments the amphipathic material is as herein disclosed and/or exemplified.

In some embodiments the Dead Sea Extract is as herein disclosed and/or exemplified.

In some embodiments the oil is a vegetable oil.

In some embodiments the oil is a cosmetic oil.

In some embodiments the cosmetic oil is selected from Octyl palmitate, Caprylic/Capric Triglyceride or Isodecyl Isononanoate.

Any other cosmetic oils are within the scope of the present disclosure.

In some embodiments the oil solution may further comprise at least one surfactant.

In some embodiments the water solution and the oil solution are dispersed at a 1:9 ration (i.e., the 10% of the dispersed water phase and 90% of the continuous oil phase).

As may be appreciated by a person versed in the art, in the methods of the invention for the encapsulation of at least one Dead Sea Extract, any dispersions means may be utilized to disperse the aqueous solution into the oil solution. See for example [2], [3], [4], EP Application No. 17306354.6 and WO 2019/068936, the content of each is incorporated herein by reference. Non limiting examples of such means are one or more of sonication means, high pressure homogenization means and tubular flow membrane contactors means.

In some embodiments the dispersion means is sonication.

In some embodiments 10% of the dispersed water phase and 90% of the continuous oil phase are dispersed utilizing sonication means.

In some embodiments sonication was conducted at pH 5.5.

In some embodiments the method provides the nanocapsules according to the invention.

Yet, in a further one of its aspects the present invention provides a method for the encapsulation of at least one Dead Sea Extract, the method comprising:

providing a water solution comprising at least one Dead Sea Extract;

providing an oil solution comprising an amphipathic material of the form A-L-B or B-L-A, wherein A is an hydrophilic polymer, B is an hydrophobic polymer and L is a linker segment or a chemical bond associating between A and B, wherein said oil solution is at a pH of between about 4.0 (inclusive) to about 6.0 (inclusive);

applying means to disperse the aqueous solution into the oil solution to thereby obtain a nanocapsule comprising a core and a shell, wherein said core comprises the at least one Dead Sea Extract and wherein said shell comprises the amphipathic material which is substantially continuously assembled around the core.

In some embodiments the water solution may optionally further comprise at least one another material such as a carrier, diluent, an excipient, a surfactant and the like, or any combination thereof.

In some embodiments the at least one another material may be a non-active and/or an active material.

In some embodiments the water solution may optionally further comprise at least one another active material e.g. as herein described.

In some embodiments the water solution may optionally further comprise at least one steric stabilizing agent.

In some embodiments the core of the nanocapsule may optionally further comprise at least one another material such as a carrier, diluent, an excipient, a surfactant and the like, or any combination thereof.

In some embodiments the at least one another material may be a non-active and/or an active material.

In some embodiments the core of the nanocapsule comprises a single droplet of water comprising the at least one Dead Sea Extract.

In some embodiments the core of the nanocapsule comprises a single droplet of Dead Sea water.

In some embodiments the core of the nanocapsule may optionally further comprise at least one another active material e.g. as herein described.

In some embodiments the amphipathic material is as herein disclosed and/or exemplified.

In some embodiments the Dead Sea Extract is as herein disclosed and/or exemplified.

In some embodiments the oil solution is at a pH of between about 4.0 (inclusive) to about 6.0 (inclusive). Any value which is between any one of pH 4.0 and pH 6.0 is within the scope of the present disclosure.

In some embodiments the oil solution is at a pH of 4.0, 4.1, 4.2, 4.3, 4.4, 4.5, 4.6, 4.7, 4.8, 4.9, 5.0, 5.1, 5.2, 5.2, 5.3, 5.4, 5.5, 5.6, 5.7, 5.8, 5.9 or 6.0. In some embodiments the oil solution is at a pH of about 5.5.

In some embodiments the oil is a vegetable oil.

In some embodiments the oil is a cosmetic oil.

In some embodiments the cosmetic oil is selected from Octyl palmitate, Caprylic/Capric Triglyceride or Isodecyl Isononanoate.

Any other cosmetic oils are within the scope of the present disclosure.

In some embodiments the oil solution may further comprise at least one surfactant.

In some embodiments the water solution and the oil solution are dispersed at a 1:9 ration (i.e., the 10% of the dispersed water phase and 90% of the continuous oil phase).

In some embodiments the dispersion means is sonication.

In some embodiments 10% of the dispersed water phase and 90% of the continuous oil phase are dispersed utilizing sonication means. In some embodiments sonication was conducted at pH 5.5.

In some embodiments the dispersion means are as herein disclosed and exemplified.

In some embodiments the method provides the nanocapsules according to the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein, the singular form “a”, “an” and “the” include plural references unless the context clearly dictates otherwise. For example, the term “an extract” or “at least one extract” may independently include a plurality of extracts, including a variety thereof.

Various embodiments and aspects of the present invention as delineated hereinabove and as claimed in the claims section below find experimental support in the following examples.

### DETAILED DESCRIPTION OF EMBODIMENTS

The following examples are not in any way intended to limit the scope of the invention as claimed.

### Example 1: Dead Sea Extract

In the present disclosure a commercial preparation of a Dead Sea extract referred to herein as “Osmoter” or “Osmoter™” or “Mineral Skin Osmoter” was used. The preparations is also known as “Maris Sal” or “Maris Aqua” (Dead Sea Water, DSW) (Source: Geological Survey - Ministry of National Infrastructures, State of Israel, especially for AHAVA-Dead Sea Laboratories CAS# INCI Monograph ID:11089).

The “Osmoter” solution has the following composition:

| | Salt normality (N) | | Salt normality (N) | | |
|---|---|---|---|---|---|
| Na | 0.118 | (2.720 g/l) | Rb | 3.5 × 10⁻⁶ | (<3 × 10⁻⁴ g/l) |
| K | 0.054 | (2.100 g/l) | Sb | <1.6 × 10⁻⁷ | (<2 × 10⁻⁵ g/l) |
| Ca | 0.873 | (35.000 g/l) | Sr | 7.6 × 10⁻³ | (0.670 g/l) |
| Mg | 3.815 | (92.700 g/l) | V | <7.9 × 10⁻⁵ | (<0.004 g/l) |
| Ba | 6.6 × 10⁻⁵ | (0.009 g/l) | Th | <8.6 × 10⁻⁸ | (<2 × 10⁻⁵ g/l) |
| Cd | <1.8 × 10⁻⁷ | (<2 × 10−⁵ g/l) | U | <8.4 × 10⁻⁸ | (<2 × 10⁻⁵ g/l) |
| Co | <3.4 × 10⁻⁵ | (<0.002 g/l) | Zn | <3.06 × 10⁻⁵ | (<0.002 g/l) |
| Cu | <3.15 × 10⁻⁵ | (<0.004 g/l) | Cl | 9.75 | (346 g/l) |
| Cr | <3.85 × 10⁻⁴ | (<0.02 g/l) | Br | 0.175 | (14 g/l) |
| Fe | <3.58 × 10⁻⁵ | (<0.002 g/l) | B | 0.011 | (0.120 g/l) |
| Li | 5.76 × 10⁻³ | (0.040 g/l) | As | 2.7 × 10⁻⁵ | (0.002 g/l) |
| Mn | 1.82 × 10⁻⁴ | (0.010 g/l) | I | 6.30 × 10⁻⁷ | (8 × 10⁻⁸ g/l) |
| Mo | <1.04 × 10⁻⁶ | (<10⁻⁴ g/l) | SiO2 | <3.33 × 10⁻⁴ | (<0.02 g/l) |
| Ni | <3.4 × 10⁻⁵ | (<0.002 g/l) | SiO4 | <2.2 × 10⁻³ | (<0.2 g/l) |
| Pb | <9.6 × 10⁻⁸ | (<2 × 10⁻⁵) | | | |

Solutions comprising Dead Sea Water were prepared by dilutions of the “Osmoter” preparation (See below). The percentages of the Dead Sea extract in the solutions are provided in weight per weight ratio (w/w) i.e., the weight in grams of the Dead Sea extract (e.g., Osmoter) per 100 gram total weight of the solution.

### Example 2: The Amphipathic Material

The amphipathic material exemplified herein was based on a polyglutamic acid (PGA) block as hydrophobic polymer at low pH (5.5) and poly(ethylene glycol) (PEG) block as the hydrophilic polymer.

The HLB of the amphipathic material was designed by varying the degree of polymerization of the polymeric blocks that build it. The degree of polymerization of the PGA block was varied from 40 to 160. PVGLIG peptide was incorporated into the diblock copolymer. Separately, some GA units were replaced by the corresponding number of Valine unit (Val), an hydrophobic peptide, to obtain non-pH-responsive diblock copolymers.

The following nanocapsules were synthesized and used in the examples below:

Non-responsive nanocapsules: the amphipathic material was prepared by replacing 30 mol % of the GA units by hydrophobic Valine units (VAL). This amount of hydrophobic co-polypeptide was found sufficient to avoid desorption of the PGA block from the O/W interface and produce stable nanocapsules at all pH.

Dual-responsive nanocapsules: In order to provide more specific biological response, especially to matrix metalloproteases MMP-2 and MMP-9, copolymers with a specific peptide sequence, namely Pro-Val-Gly-Leu-Iso-Gly (noted PVGLIG) were synthesized. First, PEG was functionalized to the peptide sequence. The resulting polymer was used as macro-initiator for the controlled Ring Opening Polymerization of BLG (benzyl-L-glutamate) followed by a de-protection step by hydrolysis to obtain the PGA block. The presence of the PGA block added a pH responsive character to this polypeptide diblock copolymer.

Enzyme-responsive nanocapsules: the amphipathic material was designed by combining both procedures used before. First, PEG homo-polymer was functionalized with the same peptide sequence. The resulting polymer was used as macro-initiator for the controlled Ring Opening copolymerization of BLG and with 30 mol % of VAL. After a de-protection step, the final di-block copolymer was obtained.

pH responsive nanocapsules: the di-block copolymer (PEG-PGA) amphipathic material was prepared.

### Example 3: Formation of the Nanocapsules

Two manufacturing processes based on mini-emulsion technique were used, namely high pressure homogenizer (HPH) and tubular flow membrane contactors (TFMC). HPH and TFMC allow controlled and reliable nano-capsules to be prepared.

### High Pressure Homogenizer

High pressure homogenizers was used for industrial scale as large capacity (21,000 L/h at 400 bar). HPHs are available and they allow obtaining high solids content dispersions of nano-droplets even for systems dealing with highly viscous organic phases [2]-[3].

### Tubular Flow Membrane Contactors

The technique uses a microporous membrane in a tubular or hollow fiber shape. The first phase flows inside the membrane tube, and the other phase flows through the membrane pores. According to the properties of both phases, in particular the miscibility of the second phase in the first one, emulsions can be obtained or colloids formed upon reaction between the two phases. The main advantage of using tubular or hollow fiber membrane geometry is the ease to increase the membrane area [4].

Nano-capsules were assembled by sonication at pH 5.5 which is the pH of healthy skin.

### Example 4: Biological Results

The objective of the current study was to evaluate the effect of various nano-capsules with Dead Sea water extract on human skin explants (ex vivo).

Ex vivo human skin organ culture (HSOC) model was used.

UVB irradiation was used to induce stress and to mimic pre-mature aging of the skin.

The following parameters were tested:
5-dimethylthiazol-2-yl]-2,5-diphenyl tetrazolium bromide (MTT) assay for epidermal viability;
Caspase 3 activity for cell apoptosis; and
TNFα secretion for inflammatory response.

The Osmoter concentration used in the capsules was 7.1%.

The Osmoter concentration in the non-encapsulated solution was 7.1% w/w (diluted with water).

The tested polymers in the Nano-capsules (NC) were as follows:

Dual—Dual responsive NC (Enzymatic and pH-responsive) comprised of the di-block copolymer (PEG-PVGLIG-PGA);

Enzyme—Enzymatic responsive NC comprised of the di-block copolymer PEG-PVGLIG-P(GA-VAL);

pH—pH-responsive NC comprised of the di-block copolymer (PEG-PGA); and

Non—Non-responsive NC comprised of the di-block copolymer [PEG-P(GA-VAL)].

### Viability Test

UVB exposure was utilized as a stressor on human skin organ culture.

Day 1: Topical application of 3 μl of each solution on the epidermis of each explant. Incubate for 24 hours.

Day 2: explant was washed once with phosphate buffer (PBS) and irradiate with UVB lamp at 200 mJ/cm2. After radiation it was incubated for 30 min and then each treatment was once again topically applied. The control sample was one not irradiated under UVB light. UVB radiated control was one without any treatment except radiation.

Day 3: Viability and caspase3 activity were measured for the epidermis layer. The medium was collected for TNFα cytokine.

The epidermal viability by MTT assay was conducted 24 hours after the UVB radiation.

The tested samples are detailed in Table 2.

TABLE 2
provides a list of tested samples*

| | Sample | | |
|---|---|---|---|
| Sample name | Identification | Cat/Lot# | UVB |
| Control | no treatment- no | | − |
| | radiation | | |
| UVB 200 mJ/cm2 | Radiation with | | + |
| | UVB | | |
| OSM-NC-Dual | OSMOTER W/O | NCW05DU | + |
| | Dual-responsive | | |
| | NC | | |
| OSM-NC-Enzyme | OSMOTER W/O | NCW05EN | + |
| | Enzyme-responsive | | |
| | NC | | |
| OSM-NC pH | OSMOTER W/O | NCW05pH | + |
| | pH-responsive NC | | |
| OSM-NC Non | OSMOTER W/O | 26ND364 | + |
| | Non-responsive NC | | |
| Osmoter as is (no | OSMOTER pH-6 | | + |
| NC) | control | | |
| NC dual | G/O Dual - | 26ND412 | + |
| | responsive NC | | |
| | without active | | |
| NC Enzyme | O/G Enzyme- | FP26AP27 | + |
| | responsive NC | | |
| | without active | | |
| NC pH | W/O pH responsive | 26ND317D | + |
| | NC without active | | |
| NC Non | W/O Non- | 26ND369D | + |
| | responsive NC | | |
| | without active | | |

*In the table the abbreviations are as follows: NC = nanocapsules; OSM = Osmoter; Active refers to Dead Sea Extract (Osmoter); W/O refers to Water in Oil emulsion; G/O refers to Glycerol in Oil emulsion; O/G refers to Oil in Glycerol emulsion.

### Results

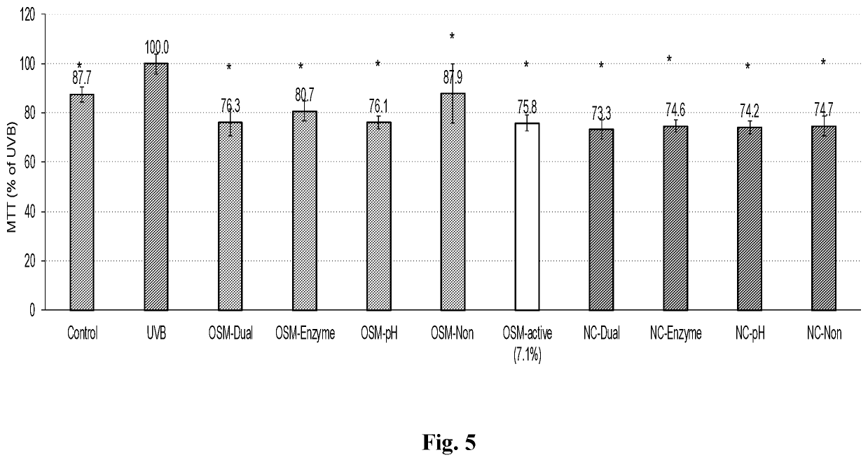

FIG. 5 illustrates the effect of topical application of Osmoter encapsulated in NC, naked NC vehicle (NC only, without the active Osmoter) and Osmoter as is, on human skin after UVB (200 mJ/cm2) radiation—viability assay (MTT). The tested polymers in the NC were as follows: Dual, Enzyme, pH and None. The observed P-value vs UVB was <0.05.

All treatments show similarity results as the control, and had a reduced in viability compare to UVB treatment.

### Conclusions

All treatments resulted in a similar value as the control. There was no advantage or disadvantage of any treatment over another.

### Apoptosis Test

The epidermal apoptosis assay was conducted 24 hour after the UVB radiation.

Apoptosis was measured as a caspase3 activity after exposing Human skin organ culture to Osmoter and Nanocapsules with Osmoter vs naked NC vehicle.

### Results

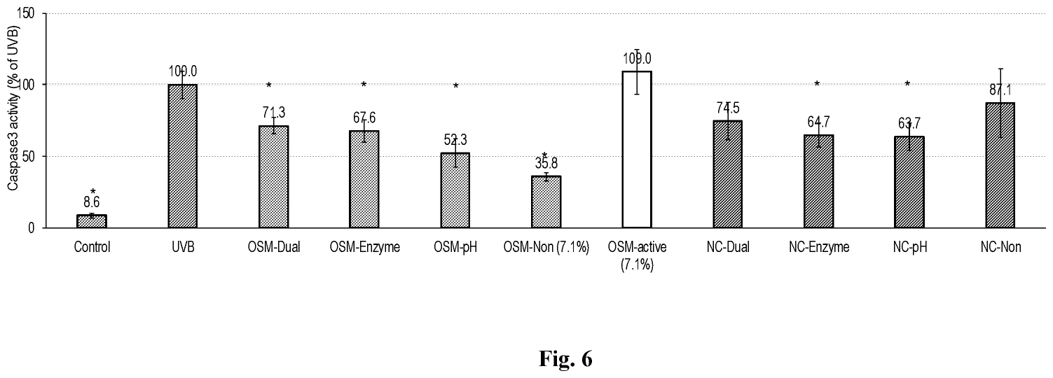

FIG. 6 illustrates the effect of topical application of Osmoter encapsulated in NC, naked NC vehicle (NC only, without the active Osmoter) and Osmoter as is on human skin after UVB (200 mJ/cm2) radiation - apoptosis activity (Caspase3 activity). The tested polymers in the NC were as follows: Dual; Enzyme; pH and None. The observed P-value vs UVB was <0.05.

The results in FIG. 6 indicate that non-encapsulated Osmoter (active), at the studied concentration i.e., 7.1%, induced apoptosis following UVB irradiation. The results further indicate that after UVB exposure the Dual-responsive NC, the enzyme responsive NC, the pH responsive NC and the non-responsive NC loaded with Osmoter showed a significant reduction in Caspase3 activity compared to the untreated skin explants.

The dual responsive NC, pH-responsive NC and non-responsive NC loaded with Osmoter showed a significant reduction in Caspase3 activity compared to the naked NC vehicle (NC only, without the active Osmoter).

### Conclusions

Encapsulated Osmoter generally showed a better protection against apoptosis induced by UVB compared to nacked capsules (NC), escpecially in non-responsive NC.

Non-encapsulated Osmoter does not have anti-apoptotic effect following UVB irradiation. However, encapsulated Osmoter provides protection against apoptosis.

### TNFα Secretion for Inflammatory Response

Epidermal TNFα secretion to the medium by HSOC model was conducted 24 hour after the UVB radiation.

The inflammatory cytokine tumor necrosis factor alpha (TNFα) secretion was measured after exposing Human skin organ culture to Osmoter and Nanocapsules (NC) with Osmoter vs naked NC vehicle.

### Results

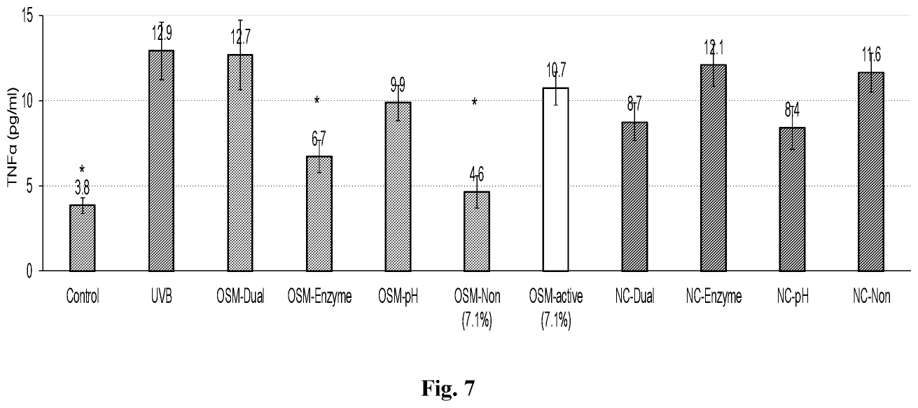

FIG. 7 illustrates the effect of topical application of Osmoter encapsulated in NC, naked NC vehicle (NC only, without the active Osmoter) and Osmoter as is, on human skin after UVB (200 mJ/cm2) radiation—secretion of TNFα to the medium. The observed P-value vs UVB was <0.05.

The results in FIG. 7 indicate that after UVB exposure, the Enzymatic-responsive NC and the non-responsive NC loaded with Osmoter showed a significant reduction in TNFα secretion compared to the untreated skin explants, the coresponding naked NC and the Osmoter as is treatment.

### Conclusions

The results indicate that non-encapsulated Osmoter (Osmoter only) does not have anti-inflammatory effect following UVB irradiation, at the studied concentration. However, encapsulated Osmoter provides protection against inflammation compared to non-encapsulated Osmoter and naked capsules (NC), in particular in the enzymatic responsive NC and the non-responsive NC.

### ILLUSTRATIVE EMBODIMENTS

The following embodiments are illustrative and not intended to limit the claimed subject matter.

Embodiment 1 A nanoparticle comprising at least one Dead Sea Extract and at least one amphipathic material of the form A-L-B, wherein:

A is an hydrophilic polymer;

B is an hydrophobic polymer; and

L is a linker segment or a chemical bond associating A to B.

Embodiment 2 The nanoparticle according to embodiment 1, wherein A is poly(ethylene glycol) (PEG) being hydrophilic at pH 5.5 or below.
Embodiment 3 The nanoparticle of embodiment 1 or 2, wherein B is polyglutamic acid (PGA).
Embodiment 4 The nanoparticle of any one of embodiments 1 to 3, wherein L is a direct bond between A and B and wherein said amphipathic material is a diblock copolymer.
Embodiment 5 The nanoparticle of embodiment 4, wherein the diblock co-polymer is a polymer wherein A is PEG and B is polyglutamic acid PGA (referred to herein as PEG-PGA).
Embodiment 6 The nanoparticle of any one of embodiments 1 to 5 wherein B is a copolymer of PGA.
Embodiment 7 The nanoparticle of embodiment 6, wherein the copolymer of PGA is a copolymer of PGA and Val [referred to herein as P(GA-VAL)].
Embodiment 8 The nanoparticle of embodiment 7, wherein the Val units in the copolymer of PGA and Val constitute at least 30% of said copolymer.
Embodiment 9 The nanoparticle of embodiment 7 or embodiment 8, wherein the copolymer of GA and Val is a diblock co-polymer.
Embodiment 10 The nanoparticle of any one of embodiments 1 to 9, wherein the number of repeating units of polymer A is between about 4 to about 100.
Embodiment 11 The nanoparticle of embodiment 10, wherein the number of repeating units of polymer A is between about 10 to about 50.
Embodiment 12 The nanoparticle of embodiment 10 or 11 wherein polymer A is PEG.
Embodiment 13 The nanoparticle of embodiment 12, wherein the repeating units of PEG is about 44.
Embodiment 14 The nanoparticle of any one of embodiments 1 to 13, wherein the number of repeating units of polymer B is between about 20 to about 100.
Embodiment 15 The nanoparticle of embodiment 14, wherein the number of repeating units of polymer B is between about 40 to 100.
Embodiment 16 The nanoparticle of embodiment 14 or embodiment 15, wherein polymer B is PGA.
Embodiment 17 The nanoparticle of any one of embodiments 1 to 16, wherein A is PEG and B is PGA or a diblock copolymer of GA and Val.
Embodiment 18 The nanoparticle of any one of embodiments 1 to 17, wherein L is a non-active linker molecule.
Embodiment 19 The nanoparticle of any one of embodiments 1 to 18, wherein L is a chemical bond directly connecting between A and B.
Embodiment 20 The nanoparticle of embodiment 19, wherein the chemical bond is selected from a C—C single bond, a C—N single bond or an amide bond.
Embodiment 21 The nanoparticle of embodiment 20, wherein the atoms taking part in said bonds are originated from polymer A and/or polymer B.
Embodiment 22 The nanoparticle of any one of embodiments 1 to 18, wherein L is a peptide unit comprising one or more amino acids, each of which may be optionally substituted.
Embodiment 23 The nanoparticle of embodiment 22, wherein the peptide unit is connected to A and B via an amide bond formed between the N or C terminus of the peptide with either A or B.
Embodiment 24 The nanoparticle of embodiment 22, wherein the peptide unit is connected to A and/or B via a side chain in the peptide, optionally a substituted side chain.
Embodiment 25 The nanoparticle of any one of embodiments 1 to 24, wherein L is the peptide PVGLIG (SEQ ID No. 1).
Embodiment 26 The nanoparticle of any one of embodiments 1 to 24, wherein L is the peptide βA-PVGLIG-βA-C (SEQ ID No. 2), optionally wherein the Cysteine is substituted.
Embodiment 27 The nanoparticle of embodiment 26, wherein L is connected to B via a peptide bond and to A via the substituted Cysteine.
Embodiment 28 The nanoparticle of embodiment 26 wherein L is the peptide having the following structure:

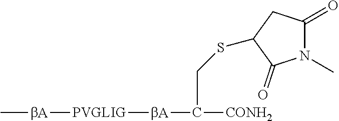

and wherein the peptide is connected to B via a peptide bond with the β-Alanine and to A via a N—C bond with the substituted cysteine moiety.
Embodiment 29 The nanoparticle of any one of embodiments 1 to 28, wherein the amphipathic material is selected to undergo dissociation in the presence of at least one Cathepsin B enzyme.
Embodiment 30 The nanoparticle of any one of embodiments 1 to 29, wherein the amphipathic material is selected to undergo dissociation at pH of between about 6.2 to 7.4.
Embodiment 31 The nanoparticle according to embodiment 29 or 30, wherein the amphipathic material is PEG-PGA (i.e., A=PEG, L=absent and B=PGA).
Embodiment 32 The nanoparticle of any one of embodiments 1 to 30, wherein the linker L is selected to undergo dissociation from either polymer A and/or polymer B when in contact with an environment having a presence of one or more of matrix metalloproteinase MMP2 and MMP9.
Embodiment 33 The nanoparticle of embodiment 32, wherein the amphipathic material is PEG-PVGLIG-P(GA-VAL) [i.e., A=PEG, L=PVGLIG and B=P(GA-VAL)].
Embodiment 34 The nanoparticle of any one of embodiments 30 to 33, wherein the amphipathic material is PEG-PVGLIG-PGA (i.e., A=PEG, L=PVGLIG and B=PGA).
Embodiment 35 The nanoparticle of any one of embodiments 1 to 34, wherein the amphipathic material is a self-assembled material forming a continuous structure.
Embodiment 36 The nanoparticle of any one of embodiments 1 to 36, wherein said nanoparticle is a nanocarrier.
Embodiment 37 The nanoparticle of embodiment 36, wherein the Dead Sea extract is comprised in the nanocarrier matrix making up the nanoparticle.
Embodiment 38 The nanoparticle of embodiment 36 or 37, wherein said nanocarrier further comprises at least one another material selected from a carrier, diluent, an excipient, a surfactant or any combination thereof.
Embodiment 39 The nanoparticle of any one of embodiments 1 to 36, wherein said nanoparticle is a nanocapsule.
Embodiment 40 The nanoparticle of embodiment 39, wherein said nanocapsule having a shell of a material and a core, wherein said core comprised the Dead Sea extract, optionally diluted in water, and wherein said shell is comprised of the amphipathic material.
Embodiment 41 The nanoparticle of embodiment 40, wherein said core further comprises at least one another material such as a carrier, diluent, an excipient, a surfactant or any combination thereof.
Embodiment 42 The nanoparticle of embodiment 38 or 41, wherein the at least one another material may be a non-active and/or an active material.
Embodiment 43 The nanoparticle of any one of embodiments 1 to 42, further comprises at least one steric stabilizing agent.
Embodiment 44 The nanoparticle of any one of embodiments 1 to 43 having a colloidal structure.
Embodiment 45 The nanoparticle of any one of embodiments 1 to 43 having a substantially spherical shape.
Embodiment 46 The nanoparticle of any one of embodiments 1 to 45 having a size of between about 1 to about 200 nm.
Embodiment 47 The nanoparticle of embodiment 46, having a size of between about 40 to 100 nm.
Embodiment 48 The nanoparticle of any one of embodiments 1 to 47, wherein said Dead Sea extract is a mixture of natural materials obtained from the waters of the Dead Sea, the mud surrounding the Dead Sea and/or the soil bed of the Dead Sea.
Embodiment 49 The nanoparticle of any one of embodiments 1 to 47, wherein said Dead Sea extract is the saline waters obtained from the Dead Sea.
Embodiment 50 The nanoparticle of embodiment 49, wherein the Dead Sea water has a specific density of 1.25-1.35 g/ml, pH of 4.6-5.6 (at 25° C.), and less than 100 cfu/g of non-pathogenic microbes.
Embodiment 51 The nanoparticle of any one of embodiments 48 to 50, wherein the Dead Sea water comprises Ca⁺², Cl⁻, Mg⁺², Na⁺, K⁺ and Br⁻.
Embodiment 52 The nanoparticle of any one of embodiments 1 to 47, wherein said Dead Sea extract is a an aqueous solution simulating the salts and minerals content of the Dead Sea water.
Embodiment 53 The nanoparticle of any one of embodiments 1 to 52, wherein said at least one Dead Sea extract is an extract as herein described and exemplified.
Embodiment 54 The nanoparticle of any one of embodiments 1 to 53, wherein the concentration of the Dead Sea extract in the nanoparticles of the invention is at least about 0.01% (w/w).
Embodiment 55 The nanoparticle of embodiment 54, wherein the concentration of the Dead Sea extract in the nanoparticles of the invention is between about 0.01% to about 7.5%.
Embodiment 56 The nanoparticle of any one of embodiment 1 to 55, wherein said nanoparticle has a core/shell structure and wherein the Dead Sea extract is present in the core of the nanoparticle in its pure form.
Embodiment 57 The nanoparticle of embodiment 56, wherein the Dead Sea extract forms the core of the nanoparticle.
Embodiment 58 The nanoparticle of any one of embodiments 1 to 58, wherein the Dead Sea extract is present in the nanoparticle in combination with at least one another active material.
Embodiment 59 The nanoparticle of embodiment 58, wherein the combination provides a synergistic effect.
Embodiment 60 The nanoparticle of embodiment 58 or 59, wherein the Dead Sea extract is present in the nanoparticle in combination with one or more of:

a) Vitamin C, at least one anti-oxidant and Hyaluronic acid;

b) Vitamin D, at least one retinoid and Hyaluronic acid;

c) Vitamin C, Curcumin and nitorxide;

d) Vitamin C;

e) Vitamin D;

f) Hyaluronic;

g) at least one Niacinamide;

h) at least one Nitroxides;

i) at least one Dipotassium glycyrrhizinate; and

j) at least one Curcumin.

Embodiment 61 An emulsion comprising a plurality of the nanoparticles according to any one of embodiments 1 to 60.
Embodiment 62 The emulsion of embodiment 61, being a water in oil (W/O) emulsion.
Embodiment 63 The emulsion of embodiment 62, wherein said oil is a cosmetic oil selected from Octyl palmitate, Caprylic/Capric Triglyceride or Isodecyl Isononanoate.
Embodiment 64 A delivery system for the delivery of at least one Dead Sea Extract to at least one skin region wherein the system comprises the nanoparticle of any one of embodiments 1 to 60 or the emulsion of any one of embodiments 61 to 63.
Embodiment 65 The delivery system of embodiment 64, wherein the amphipathic material in the nanoparticle is selected to undergo dissociation and/or wherein the linker L in the nanoparticle is selected to undergo dissociation from either polymer A and/or polymer B when in contact with skin environment having one or more of: (i) pH of between about 6.2 to 7.4; (ii) presence of one or more of matrix metalloproteinase MMP2 and MMP9; and (iii) presence of at least one Cathepsin B enzyme, wherein said dissociation induced the degradation of the amphipathic material and the release of the active ingredient to said skin environment.
Embodiment 66 The delivery system of embodiment 64, wherein the nanoparticle has a core/shell structure, wherein the Dead Sea extract is comprised within said core, and wherein the shell material comprises the amphipathic material, wherein the amphipathic material is selected to undergo dissociation and/or wherein the linker L in the nanoparticle is selected to undergo dissociation from either polymer A and/or polymer B when in contact with skin environment having one or more of: (i) pH of between about 6.2 to 7.4; (ii) presence of one or more of matrix metalloproteinase MMP2 and MMP9; and (iii) presence of at least one Cathepsin B enzyme, wherein said dissociation induced the degradation of the shell material and the release of the Dead Sea extract from the core to said skin environment.
Embodiment 67 The delivery system of any one of embodiments 64 to 66, wherein said skin environment is associated with a disease or a disorder selected from one or more of irritant contact dermatitis (ICD), allergic contact dermatitis (ACD), skin photo-aging and skin pigmentation.
Embodiment 68 A composition comprising the nanoparticle of any one of embodiments 1 to 60, the emulsion of any one of embodiments 61 to 63, or the delivery system of any one of embodiments 64 to 67.
Embodiment 69 The composition of embodiment 68, being a skin-care composition and/or a pharmaceutical compositions.
Embodiment 70 The composition of embodiment 68 or embodiment 69, wherein the Dead Sea extract is present in said composition at a concentration of at least about 0.01% (w/w).
Embodiment 71 The composition of embodiment 70, wherein the Dead Sea extract is present in said composition at a concentration of between about 0.01% to about 7.5%.
Embodiment 72 The nanoparticle of any one of embodiments 1 to 60, for use in the preparation of skin-care and/or pharmaceutical formulations.
Embodiment 73 The nanoparticle of any one of embodiments 1 to 60, the emulsion of any one of embodiments 61 to 63, or the delivery system of any one of embodiments 64 to 67 for one or more of protecting and/or improving the state of the skin, and preventing and/or treating imperfections of the skin of a subject.
Embodiment 74 The nanoparticle of any one of embodiments 1 to 60, the emulsion of any one of embodiments 61 to 63, or the delivery system of any one of embodiments 64 to 67 for treating or preventing at least one disease or disorder of the skin.
Embodiment 75 Use of the nanoparticle of any one of embodiments 1 to 60, the emulsion of any one of embodiments 61 to 63, or the delivery system of any one of embodiments 64 to 67 for the preparation of a pharmaceutical composition for treating or preventing a disease or disorder of the skin.
Embodiment 76 The use of embodiment 75, wherein the disease or disorder are associated with skin environment having one or more of (i) pH of between about 6.2 to 7.4; (ii) presence of one or more of matrix metalloproteinase MMP2 and MMP9; and (iii) presence of at least one Cathepsin B enzyme.
Embodiment 77 The use of embodiment 76, wherein the disease or disorder are selected from irritant contact dermatitis (ICD), allergic contact dermatitis (ACD), skin photo-aging and skin pigmentation.
Embodiment 78 A lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, and an eye make-up comprising the nanoparticle of any one of embodiments 1 to 60, the emulsion of any one of embodiments 61 to 63, or the delivery system of any one of embodiments 64 to 67.
Embodiment 79 A method for one or more of protecting and/or improving the state of the skin of a subject and preventing and/or treating imperfections of the skin of a subject in need thereof, wherein the method comprises topically administering the nanoparticle of any one of embodiments 1 to 60, the emulsion of any one of embodiments 61 to 63, or the delivery system of any one of embodiments 64 to 67 onto the skin of the subject.
Embodiment 80 A method for treating or preventing a disease or disorder of the skin of a subject, the method comprises administering to a subject in need thereof the nanoparticle of any one of embodiments 1 to 60, the emulsion of any one of embodiments 61 to 63, or the delivery system of any one of embodiments 64 to 67 onto the skin of the subject.
Embodiment 81 The method of embodiment 80, wherein the disease or disorder are associated with skin environment having one or more of (i) pH of between about 6.2 to 7.4; (ii) presence of one or more of matrix metalloproteinase MMP2 and MMP9; and (iii) presence of at least one Cathepsin B enzyme.
Embodiment 82 The method of embodiment 81, wherein the disease or disorder are selected from irritant contact dermatitis (ICD), allergic contact dermatitis (ACD), skin photo-aging and skin pigmentation.

The following further embodiments are illustrative and not intended to limit the claimed subject matter.

Embodiment 1A A nanoparticle comprising at least one Dead Sea Extract and at least one amphipathic material of the form A-L-B or B-L-A, wherein:

A is an hydrophilic polymer;

B is an hydrophobic polymer; and

L is a linker segment or a chemical bond associating between A and B.

Embodiment 2A The nanoparticle of embodiment 1A, wherein a combination of A, L and B in said amphipathic material is configured to provide an amphipathic material, wherein said amphipathic material is configured to undergo dissociation in environment having one or more of: (i) pH of between about 6.2 to 7.4; (ii) presence of one or more of matrix metalloproteinase MMP2 and MMP9; and (iii) presence of at least one Cathepsin B enzyme.
Embodiment 3A The nanoparticle of embodiment 1A or 2A, wherein the amphipathic material is selected to undergo dissociation and/or wherein the linker L in the amphipathic material is selected to undergo dissociation from either polymer A and/or polymer B in environment having one or more of: (i) pH of between about 6.2 to 7.4; (ii) presence of one or more of matrix metalloproteinase MMP2 and MMP9; and (iii) presence of at least one Cathepsin B enzyme.
Embodiment 4A The nanoparticle of any one of embodiments 1A to 3A, wherein the amphipathic material is selected to undergo dissociation in the presence of at least one Cathepsin B enzyme.
Embodiment 5A The nanoparticle of any one of embodiments 1A to 4A, wherein the amphipathic material is selected to undergo dissociation at pH of between about 6.2 to 7.4.
Embodiment 6A The nanoparticle of any one of embodiments 1A to 5A, wherein the linker L is selected to undergo dissociation from either polymer A and/or polymer B when in contact with an environment having a presence of one or more of matrix metalloproteinase MMP2 and MMP9.
Embodiment 7A The nanoparticle of any one of embodiments 2A to 6A, wherein said environment is a skin environment.
Embodiment 8A The nanoparticle of any one of embodiments 1A to 7A, wherein A is poly(ethylene glycol) (PEG).
Embodiment 9A The nanoparticle of any one of embodiments 1A to 8A, wherein B is polyglutamic acid (PGA).
Embodiment 10A The nanoparticle of any one of embodiments 1A to 9A, wherein L is a direct bond between A and B and wherein said amphipathic material is a diblock copolymer.
Embodiment 11A The nanoparticle of embodiment 10A, wherein the diblock co-polymer is a polymer wherein A is PEG and B is polyglutamic acid PGA.
Embodiment 12A The nanoparticle of any one of embodiments 1A to 11A wherein B is a copolymer of PGA.
Embodiment 13A The nanoparticle of embodiment 12A, wherein the copolymer of PGA is a copolymer of PGA and Val.
Embodiment 14A The nanoparticle of embodiment 13A, wherein the Val units in the copolymer of PGA and Val constitute at least 30% of said copolymer.
Embodiment 15A The nanoparticle of embodiment 13A or 14A, wherein the copolymer of GA and Val is a diblock co-polymer.
Embodiment 16A The nanoparticle of any one of embodiments 1A to 15A, wherein the number of repeating units of polymer A is between about 4 to about 120.
Embodiment 17A The nanoparticle of any one of embodiments 1A to 16A wherein polymer A is PEG.
Embodiment 18A The nanoparticle of any one of embodiments 1A to 17A, wherein the number of repeating units of polymer B is between about 20 to about 160.
Embodiment 19A The nanoparticle of embodiment 18A, wherein polymer B is PGA.
Embodiment 20A The nanoparticle of any one of embodiments 1A to 19A, wherein A is PEG and B is PGA or a diblock copolymer of GA and Val.
Embodiment 21A The nanoparticle of any one of embodiments 1A to 20A, wherein L is a non-active linker molecule.
Embodiment 22A The nanoparticle of any one of embodiments 1A to 21A, wherein L is a chemical bond directly connecting between A and B.
Embodiment 23A The nanoparticle of embodiment 22A, wherein the chemical bond is selected from a C—C single bond, a C—N single bond or an amide bond.
Embodiment 24A The nanoparticle of embodiment 23A, wherein the atoms taking part in said bonds are originated from polymer A and/or polymer B.
Embodiment 25A The nanoparticle of any one of embodiments 1A to 21A, wherein L is a peptide unit comprising one or more amino acids, each of which may be optionally substituted.
Embodiment 26A The nanoparticle of embodiment 25A, wherein the peptide unit is connected to A and B via an amide bond formed between the N or C terminus of the peptide with either A or B.
Embodiment 27A The nanoparticle of embodiment 25A, wherein the peptide unit is connected to A and/or B via a side chain in the peptide, optionally a substituted side chain.
Embodiment 28A The nanoparticle of any one of embodiments 1A to 27A, wherein L is the peptide PVGLIG (SEQ ID No. 1).
Embodiment 29A The nanoparticle of any one of embodiments 1A to 27A, wherein L is the peptide βA-PVGLIG-βA-C (SEQ ID No. 2), optionally wherein the Cysteine is substituted.
Embodiment 30A The nanoparticle of embodiment 29A, wherein L is connected to B via a peptide bond and to A via the substituted Cysteine.
Embodiment 31A The nanoparticle of embodiment 29A wherein L is the peptide having the following structure:

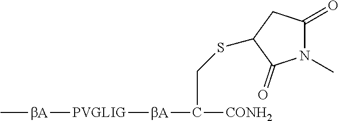

and wherein the peptide is connected to B via a peptide bond with the β-Alanine and to A via a N—C bond with the substituted cysteine moiety.
Embodiment 32A The nanoparticle of any one of embodiments 1A to 31A, wherein L is selected from the peptide PVGLIG (SEQ ID No. 1), the peptide βA-PVGLIG-βA-C (SEQ ID No. 2) (wherein C is optionally substituted) or the peptide having the following structure:

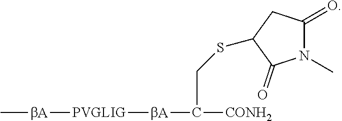

Embodiment 33A The nanoparticle of any one of embodiments 1A to 32A wherein said amphipathic material is of the form A-L-B or B-L-A, wherein:

A is an hydrophilic polymer (PEG)X, wherein x is between about 4 to about 120;

B is an hydrophobic polymer P(GA_{y}-Val_{z}), wherein y is between about 20 to about 160 and z is 0 or at least about 30% of the value of y; and

L is a linker segment or a chemical bond associating between A and B.

Embodiment 34A The nanoparticle of embodiment 33A, wherein x is 114, y is 150 or 151 and z is 0.
Embodiment 35A The nanoparticle of embodiment 33A, wherein x is 114, y is 46 and z is 11.
Embodiment 36A The nanoparticle of any one of embodiments 33A to 35A, wherein L is a peptide or a chemical bond associating between A and B.
Embodiment 37A The nanoparticle of embodiment 36A, wherein L is a peptide selected from PVGLIG (SEQ ID No. 1), βA-PVGLIG-βA-C (SEQ ID No. 2) (wherein C is optionally substituted) or a peptide having the following structure:

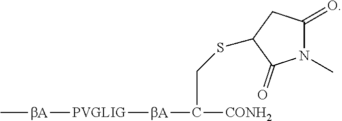

Embodiment 38A The nanoparticle of embodiment 37A, wherein L is the peptide PVGLIG (SEQ ID No. 1).
Embodiment 39A The nanoparticle of any one of embodiments 1A to 38A, wherein the amphipathic material is a copolymer of formula selected from the group consisting of:

(i) PEGₓ-PGA_{y} wherein x is between about 4 to about 120 and y is between about 20 to about 160;

(ii) PGA_{y}-PVGLIG-PEGₓ wherein y is between about 20 to about 160 and x is between about 4 to about 120; and

(iii) PEGₓ-PVGLIG-P(GA_{y}-Val_{z}) wherein x is between about 4 to about 120, y is between about 20 to about 160 and z is at least about 30% of the value of y.

Embodiment 40A The nanoparticle of embodiment 39A, wherein the amphipathic material is PEGₓ-PGA_{y} wherein x is between about 4 to about 120 and y is between about 20 to about 160.
Embodiment 41A The nanoparticle of embodiment 40A, wherein the amphipathic material is selected to undergo dissociation in environment having one or more of (i) pH of between about 6.2 to 7.4; and (ii) presence of at least one Cathepsin B enzyme.
Embodiment 42A The nanoparticle of embodiment 40A or 41A, wherein the amphipathic material is PEG₁₁₄-PGA₁₅₁.
Embodiment 43A The nanoparticle of embodiment 39A wherein the amphipathic material is PGA_{y}-PVGLIG-PEGₓ wherein y is between about 20 to about 160 and x is between about 4 to about 120.
Embodiment 44A The nanoparticle of embodiment 43A, wherein said amphipathic material is selected to undergo dissociation in environment having one or more of: (i) pH of between about 6.2 to 7.4; (ii) presence of one or more of matrix metalloproteinase MMP2 and MMP9; and (iii) presence of at least one Cathepsin B enzyme.
Embodiment 45A The nanoparticle of embodiment 43A or 44A, wherein the amphipathic material is PGA₁₅₀-PVGLIG-PEG₁₁₄.
Embodiment 46A The nanoparticle of embodiment 39A, wherein the amphipathic material is PEGₓ-PVGLIG-P(GA_{y}-Val_{z}) wherein x is between about 4 to about 120, y is between about 20 to about 160 and z is at least about 30% of the value of y.
Embodiment 47A The nanoparticle of embodiment 46A, wherein the amphipathic material is selected to undergo dissociation in environment having presence of one or more of matrix metalloproteinase MMP2 and MMP9.
Embodiment 48A The nanoparticle of embodiment 46A or 47A, wherein the amphipathic material is PEG₁₁₄-PVGLIG-P(GA₄₆-Val₁₁).
Embodiment 49A The nanoparticle of any one of embodiments 1A to 48A, wherein said nanoparticle is a nanocarrier.
Embodiment 50A The nanoparticle of embodiment 49A, wherein the Dead Sea extract is comprised in the nanocarrier matrix making up the nanoparticle.
Embodiment 51A The nanoparticle of embodiment 49A or 50A, wherein said nanocarrier further comprises at least one another material selected from a carrier, diluent, an excipient, a surfactant or any combination thereof.
Embodiment 52A The nanoparticle of any one of embodiments 1A to 48A, wherein said nanoparticle is a nanocapsule.
Embodiment 53A The nanoparticle of embodiment 52A, wherein said nanocapsule having a shell of a material and a core, wherein said core comprised the Dead Sea extract, optionally diluted in water, and wherein said shell is comprised of the amphipathic material.
Embodiment 54A The nanoparticle of embodiment 53A, wherein said core further comprises at least one another material such as a carrier, diluent, an excipient, a surfactant or any combination thereof.
Embodiment 55A The nanoparticle of embodiment MA or 54A, wherein the at least one another material may be a non-active and/or an active material.
Embodiment 56A The nanoparticle of any one of embodiments 1A to 55A, further comprises at least one steric stabilizing agent.
Embodiment 57A The nanoparticle of any one of embodiments 1A to 56A having a colloidal structure.
Embodiment 58A The nanoparticle of any one of embodiments 1A to 56A having a substantially spherical shape.
Embodiment 59A The nanoparticle of any one of embodiments 1A to 58A having a size of between about 1 to about 200 nm.
Embodiment 60A The nanoparticle of embodiment 59A, having a size of between about 40 to 100 nm.
Embodiment 61A The nanoparticle of any one of embodiments 1A to 60A, wherein said Dead Sea extract is a mixture of natural materials obtained from the waters of the Dead Sea, the mud surrounding the Dead Sea and/or the soil bed of the Dead Sea.
Embodiment 62A The nanoparticle of any one of embodiments 1A to 60A, wherein said Dead Sea extract is the saline waters obtained from the Dead Sea.
Embodiment 63A The nanoparticle of embodiment 62A, wherein the Dead Sea water has a specific density of 1.25-1.35 g/ml, pH of 4.6-5.6 (at 25° C.), and less than 100 cfu/g of non-pathogenic microbes.
Embodiment 64A The nanoparticle of any one of embodiments 61A to 63A, wherein the Dead Sea water comprises Ca⁺², Cl⁻, Mg⁺², Na⁺, K⁺ and Br⁻.
Embodiment 65A The nanoparticle of any one of embodiments 1A to 60A, wherein said Dead Sea extract is a an aqueous solution simulating the salts and minerals content of the Dead Sea water.
Embodiment 66A The nanoparticle of any one of embodiments 1A to 65A, wherein said at least one Dead Sea extract is an extract as herein described and exemplified.
Embodiment 67A The nanoparticle of any one of embodiments 1A to 66A, wherein the concentration of the Dead Sea extract in the nanoparticles of the invention is at least about 0.01% (w/w).
Embodiment 68A The nanoparticle of embodiment 67A, wherein the concentration of the Dead Sea extract in the nanoparticles of the invention is between about 0.01% to about 7.5%.
Embodiment 69A The nanoparticle of any one of embodiments 1A to 68A, wherein said nanoparticle has a core/shell structure and wherein the Dead Sea extract is present in the core of the nanoparticle in its pure form.
Embodiment 70A The nanoparticle of embodiment 69A, wherein the Dead Sea extract forms the core of the nanoparticle.
Embodiment 71A The nanoparticle of embodiment 70A, wherein the core of the nanoparticle constitutes a single drop comprising the Dead Sea extract.
Embodiment 72A The nanoparticle of any one of embodiments 1A to 71A, wherein the Dead Sea extract is present in the nanoparticle in combination with at least one another active material.
Embodiment 73A The nanoparticle of embodiment 72A, wherein the combination provides a synergistic effect.
Embodiment 74A The nanoparticle of embodiment 72A or 73A, wherein the Dead Sea extract is present in the nanoparticle in combination with one or more of:

a) Vitamin C, at least one anti-oxidant and Hyaluronic acid;

b) Vitamin D, at least one retinoid and Hyaluronic acid;

c) Vitamin C, Curcumin and nitorxide;

d) Vitamin C;

e) Vitamin D;

f) Hyaluronic acid;

g) at least one Niacinamide;

h) at least one Nitroxides;

i) at least one Dipotassium glycyrrhizinate; and

j) at least one Curcumin.

Embodiment 75A An emulsion comprising a plurality of the nanoparticles of any one of embodiments 1A to 74A.
Embodiment 76A The emulsion of embodiment 75A, being a water in oil (W/O) emulsion.
Embodiment 77A The emulsion of embodiment 76A, wherein said oil is a cosmetic oil selected from Octyl palmitate, Caprylic/Capric Triglyceride or Isodecyl Isononanoate.
Embodiment 78A A delivery system for the delivery of at least one Dead Sea Extract to at least one skin region wherein the system comprises the nanoparticle of any one of embodiments 1A to 74A or the emulsion of any one of embodiments 75A to 77A.
Embodiment 79A The delivery system of embodiment 78A, wherein the amphipathic material in the nanoparticle is selected to undergo dissociation and/or wherein the linker L in the nanoparticle is selected to undergo dissociation from either polymer A and/or polymer B when in contact with skin environment having one or more of: (i) pH of between about 6.2 to 7.4; (ii) presence of one or more of matrix metalloproteinase MMP2 and MMP9; and (iii) presence of at least one Cathepsin B enzyme, wherein said dissociation induced the degradation of the amphipathic material and the release of the active ingredient to said skin environment.
Embodiment 80A The delivery system of embodiment 78A, wherein the nanoparticle has a core/shell structure, wherein the Dead Sea extract is comprised within said core, and wherein the shell material comprises the amphipathic material, wherein the amphipathic material is selected to undergo dissociation and/or wherein the linker L in the nanoparticle is selected to undergo dissociation from either polymer A and/or polymer B when in contact with skin environment having one or more of: (i) pH of between about 6.2 to 7.4; (ii) presence of one or more of matrix metalloproteinase MMP2 and MMP9; and (iii) presence of at least one Cathepsin B enzyme, wherein said dissociation induced the degradation of the shell material and the release of the Dead Sea extract from the core to said skin environment.
Embodiment 81A The delivery system of any one of embodiments 78A to 80A, wherein said skin environment is associated with a disease or a disorder selected from one or more of irritant contact dermatitis (ICD), allergic contact dermatitis (ACD), skin photo-aging and skin pigmentation.
Embodiment 82A A composition comprising the nanoparticle of any one of embodiments 1A to 74A, the emulsion of any one of embodiments 75A to 77A, or the delivery system of any one of embodiments 78A to 81A.
Embodiment 83A The composition of embodiment 82A, being a skin-care composition and/or a pharmaceutical compositions.
Embodiment 84A The composition of embodiment 82A or embodiment 83A, wherein the Dead Sea extract is present in said composition at a concentration of at least about 0.01% (w/w).
Embodiment 85A The composition of any one of embodiments 82A to 84A, wherein the Dead Sea extract is present in said composition at a concentration of between about 0.01% to about 7.5%.
Embodiment 86A The nanoparticle of any one of embodiments 1A to 74A, for use in the preparation of skin-care and/or pharmaceutical formulations.
Embodiment 87A The nanoparticle of any one of embodiments 1A to 74A, the emulsion of any one of embodiments 75A to 77A, or the delivery system of any one of embodiments 78A to 81A for one or more of protecting and/or improving the state of the skin, and preventing and/or treating imperfections of the skin of a subject.
Embodiment 88A The nanoparticle of any one of embodiments 1A to 74A, the emulsion of any one of embodiments 75A to 77A, or the delivery system of any one of embodiments 78A to 81A for treating or preventing at least one disease or disorder of the skin.
Embodiment 89A Use of the nanoparticle of any one of embodiments 1A to 74A, the emulsion of any one of embodiments 75A to 77A, or the delivery system of any one of embodiments 78A to 81A for the preparation of a pharmaceutical composition for treating or preventing a disease or disorder of the skin.
Embodiment 90A The use of embodiment 89A, wherein the disease or disorder are associated with skin environment having one or more of (i) pH of between about 6.2 to 7.4; (ii) presence of one or more of matrix metalloproteinase MMP2 and MMP9; and (iii) presence of at least one Cathepsin B enzyme.
Embodiment 91A The use of embodiment 89A, wherein the disease or disorder are selected from irritant contact dermatitis (ICD), allergic contact dermatitis (ACD), skin photo-aging and skin pigmentation.
Embodiment 92A A lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, and an eye make-up comprising the nanoparticle of any one of embodiments 1A to 74A, the emulsion of any one of embodiments 75A to 77A, or the delivery system of any one of embodiments 78A to 81A.
Embodiment 93A A method for one or more of protecting and/or improving the state of the skin of a subject and preventing and/or treating imperfections of the skin of a subject in need thereof, wherein the method comprises topically administering the nanoparticle of any one of embodiments 1A to 74A, the emulsion of any one of embodiments 75A to 77A, or the delivery system of any one of embodiments 78A to 81A onto the skin of the subject.
Embodiment 94A A method for treating or preventing a disease or disorder of the skin of a subject, the method comprises administering to a subject in need thereof the nanoparticle of any one of embodiments 1A to 74A, the emulsion of any one of embodiments 75A to 77A, or the delivery system of any one of embodiments 78A to 81A onto the skin of the subject.
Embodiment 95A The method of embodiment 94A, wherein the disease or disorder are associated with skin environment having one or more of (i) pH of between about 6.2 to 7.4; (ii) presence of one or more of matrix metalloproteinase MMP2 and MMP9; and (iii) presence of at least one Cathepsin B enzyme.
Embodiment 96A The method of embodiment 95A, wherein the disease or disorder are selected from irritant contact dermatitis (ICD), allergic contact dermatitis (ACD), skin photo-aging and skin pigmentation.
Embodiment 97A A method for the encapsulation of at least one Dead Sea Extract, the method comprising:

providing a water solution comprising at least one Dead Sea Extract and optionally at least one amphipathic material;

providing an oil solution comprising at least one oil and optionally at least one amphipathic material;

provided that one of said water solution or oil solution comprises at least one amphipathic material of the form A-L-B or B-L-A, wherein:

A is an hydrophilic polymer;

B is an hydrophobic polymer; and

L is a linker segment or a chemical bond associating between A and B;

and being at a pH of between about 4.0 to about 6.0; and

applying means to disperse the aqueous solution into the oil solution to thereby obtain a nanocapsule comprising a core and a shell, wherein said core comprises the at least one Dead Sea Extract and wherein said shell comprises said at least one amphipathic material which is substantially continuously assembled around the core.

Embodiment 98A The method of embodiment 97A, wherein said at least one amphipathic material is substantially as disclosed herein.

1.-98. (canceled)

99. A nanoparticle comprising at least one Dead Sea Extract and at least one amphipathic material of the form A-L-B or B-L-A, wherein:
A is an hydrophilic polymer;
B is an hydrophobic polymer; and
L is a linker segment or a chemical bond associating between A and B;
wherein the amphipathic material is selected to undergo dissociation in the presence of at least one Cathepsin B enzyme and/or at a pH of between about 6.2 to 7.4, and/or wherein the linker L is selected to undergo dissociation from either polymer A and/or polymer B when in contact with an environment having a presence of one or more of matrix metalloproteinase MMP2 and MMP9.

100. The nanoparticle of claim 99, wherein A is poly(ethylene glycol) (PEG) and wherein B is polyglutamic acid (PGA) or a copolymer of PGA and Val.

101. The nanoparticle of claim 99, wherein L is a peptide unit comprising one or more amino acids, each of which may be optionally substituted.

102. The nanoparticle of claim 99, wherein L is selected from the peptide PVGLIG (SEQ ID No. 1), the peptide βA-PVGLIG-βA-C (SEQ ID No. 2) (wherein C is optionally substituted) or the peptide having the following structure:

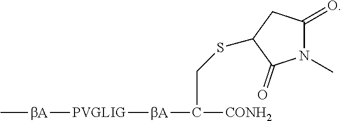

103. The nanoparticle of claim 99, wherein the amphipathic material is a copolymer of formula selected from the group consisting of:
(i) PEGₓ-PGA_{y} wherein x is between about 4 to about 120 and y is between about 20 to about 160;
(ii) PGA_{y}-PVGLIG-PEGₓ wherein y is between about 20 to about 160 and x is between about 4 to about 120; and
(iii) PEGₓ-PVGLIG-P(GA_{y}-Val_{z}) wherein x is between about 4 to about 120, y is between about 20 to about 160 and z is at least about 30% of the value of y.

104. The nanoparticle of claim 103, wherein the amphipathic material is PEGₓ-PGA_{y} wherein x is between about 4 to about 120 and y is between about 20 to about 160, and wherein the amphipathic material is selected to undergo dissociation in environment having one or more of (i) pH of between about 6.2 to 7.4; and (ii) presence of at least one Cathepsin B enzyme.

105. The nanoparticle of claim 103 wherein the amphipathic material is PGA_{y}-PVGLIG-PEGₓ wherein y is between about 20 to about 160 and x is between about 4 to about 120, and wherein said amphipathic material is selected to undergo dissociation in environment having one or more of: (i) pH of between about 6.2 to 7.4; (ii) presence of one or more of matrix metalloproteinase MMP2 and MMP9; and (iii) presence of at least one Cathepsin B enzyme.

106. The nanoparticle of claim 103, wherein the amphipathic material is PEGₓ-PVGLIG-P(GA_{y}-Val_{z}) wherein x is between about 4 to about 120, y is between about 20 to about 160 and z is at least about 30% of the value of y, and wherein the amphipathic material is selected to undergo dissociation in environment having presence of one or more of matrix metalloproteinase MMP2 and MMP9.

107. The nanoparticle of claim 99, wherein said nanoparticle is a nanocarrier, wherein the Dead Sea extract is comprised in the nanocarrier matrix making up the nanoparticle, or wherein said nanoparticle is a nanocapsule, wherein said nanocapsule having a shell of a material and a core, wherein said core comprise the Dead Sea extract, optionally diluted in water, and wherein said shell is comprised of the amphipathic material, optionally wherein said nanocarrier or said core further comprises at least one another material such as a carrier, diluent, an excipient, a surfactant or any combination thereof.

108. The nanoparticle of claim 99, wherein said Dead Sea extract is a mixture of natural materials obtained from the waters of the Dead Sea, the mud surrounding the Dead Sea and/or the soil bed of the Dead Sea.

109. The nanoparticle of claim 99, wherein the Dead Sea extract is present in the nanoparticle in combination with at least one another active material.

110. A delivery system for the delivery of at least one Dead Sea Extract to at least one skin region wherein the system comprises the nanoparticle of claim 99, wherein the amphipathic material in the nanoparticle is selected to undergo dissociation when in contact with skin environment having presence of at least one Cathepsin B enzyme and/or having a pH of between about 6.2 to 7.4, and/or wherein the linker L in the nanoparticle is selected to undergo dissociation from either polymer A and/or polymer B when in contact with skin environment having presence of one or more of matrix metalloproteinase MMP2 and MMP9, wherein said dissociation induces the degradation of the amphipathic material and the release of the active ingredient to said skin environment.

111. The delivery system of claim 110, wherein said skin environment is associated with a disease or a disorder selected from one or more of irritant contact dermatitis (ICD), allergic contact dermatitis (ACD), skin photo-aging and skin pigmentation.

112. A composition comprising the nanoparticle of claim 99.

113. A lotion, an ointment, a gel, a mask, a toner, an essence, a shampoo, a moisturizer, a sunscreen, a cream, a stick, a spray, an aerosol, foam, a paste, a mousse, a solid, semi-solid, or a liquid make-up, a foundation, and an eye make-up comprising the nanoparticle of claim 99.

114. A method for one or more of protecting and/or improving the state of the skin of a subject and preventing and/or treating imperfections of the skin of a subject in need thereof, wherein the method comprises topically administering the nanoparticle of claim 99 onto the skin of the subject.

115. A method for treating or preventing a disease or disorder of the skin of a subject, the method comprises administering to a subject in need thereof the nanoparticle of claim 99 onto the skin of the subject.

116. The method of claim 115, wherein the disease or disorder are associated with skin environment having one or more of (i) pH of between about 6.2 to 7.4; (ii) presence of one or more of matrix metalloproteinase MMP2 and MMP9; and (iii) presence of at least one Cathepsin B enzyme.

117. The method of claim 116, wherein the disease or disorder are selected from irritant contact dermatitis (ICD), allergic contact dermatitis (ACD), skin photo-aging and skin pigmentation.

118. A method for the encapsulation of at least one Dead Sea Extract, the method comprising:
providing a water solution comprising at least one Dead Sea Extract and optionally at least one amphipathic material;
providing an oil solution comprising at least one oil and optionally at least one amphipathic material;
provided that one of said water solution or oil solution comprises at least one amphipathic material of the form A-L-B or B-L-A, wherein:
A is an hydrophilic polymer;
B is an hydrophobic polymer; and
L is a linker segment or a chemical bond associating between A and B;
and being at a pH of between about 4.0 to about 6.0;
wherein said amphipathic material is selected to undergo dissociation in the presence of at least one Cathepsin B enzyme and/or at a pH of between about 6.2 to 7.4, and/or wherein the linker L is selected to undergo dissociation from either polymer A and/or polymer B when in contact with an environment having a presence of one or more of matrix metalloproteinase MMP2 and MMP9; and
applying means to disperse the aqueous solution into the oil solution to thereby obtain a nanocapsule comprising a core and a shell, wherein said core comprises the at least one Dead Sea Extract and wherein said shell comprises said at least one amphipathic material which is substantially continuously assembled around the core.


SEQUENCE LISTING (2 sequences)

SEQ ID NO: 1 - protein, 6 aa, Artificial Sequence
  Peptide 1
PVGLIG

SEQ ID NO: 2 - protein, 9 aa, Artificial Sequence
  Peptide 2
  MISC_FEATURE (1)..(1): beta Alanine
  MISC_FEATURE (8)..(8): beta Alanine
APVGLIGAC